(19) United States
(12) Patent Application Publication
(10) Pub. No.: US 2024/0400473 A1
(43) Pub. Date: Dec. 5, 2024

(54) CATALYST SYSTEM AND ETHYLENE OLIGOMERIZATION PROCESS FOR THE PREPARATION OF LINEAR ALPHA OLEFINS

(71) Applicant: Chevron Phillips Chemical Company LP, The Woodlands, TX (US)

(72) Inventors: Brooke L. Small, Kingwood, TX (US); Matthew F. Milner, Kingwood, TX (US); Michael Webster-Gardiner, Humble, TX (US); Julie A. Leseberg, Kingwood, TX (US)

(21) Appl. No.: 18/642,898

(22) Filed: Apr. 23, 2024

Related U.S. Application Data
(60) Provisional application No. 63/498,537, filed on Apr. 27, 2023.
(60) Provisional application No. 63/498,538, filed on Apr. 27, 2023.
(60) Provisional application No. 63/498,559, filed on Apr. 27, 2023.

(51) Int. Cl. C07C 2/30; C07C 2/34

(52) U.S. Cl. CPC C07C 2/30; C07C 2/34; C07C 2531/14; C07C 2531/16

(57) ABSTRACT

Catalyst compositions containing an organoaluminum compound, a hydrocarbon diluent, and a heteroatomic ligand transition metal compound complex or a heteroatomic ligand and a transition metal compound are disclosed. The transition metal is iron, cobalt, or nickel. Related ethylene oligomerization processes utilizing the catalyst compositions to produce oligomer products containing 1-hexene and 1-octene also are described.

### REFERENCE TO RELATED APPLICATIONS

This application claims the benefit of U.S. Provisional Patent Application Nos. 63/498,537, 63/498,538, and 63/498,559, filed on Apr. 27, 2023, the disclosures of which are incorporated herein by reference in their entirety.

### FIELD OF THE INVENTION

The present disclosure relates generally to a catalyst composition containing an organoaluminum compound, a hydrocarbon diluent, and a heteroatomic ligand transition metal compound complex or a heteroatomic ligand and a transition metal compound, and the use of the catalyst composition in an ethylene oligomerization process.

### BACKGROUND OF THE INVENTION

Alpha olefins such as 1-hexene and 1-octene can be produced using an ethylene reactant and various combinations of catalyst systems and oligomerization processes. It can be beneficial for the catalyst system employed to have high catalytic activity and good compatibility with other materials present in the oligomerization reactor, as well as being more selective to desirable C₆-C₈ linear α-olefins. Accordingly, it is to these ends that the present invention is generally directed.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Disclosed herein are catalyst compositions and methods for using the catalyst compositions to oligomerize olefins. In one aspect, the catalyst composition can comprise (a) an organoaluminum compound, (b) a hydrocarbon diluent, and (c) a heteroatomic ligand transition metal compound complex comprising Fe, Co, or Ni. In another aspect, the catalyst composition can comprise (A) an organoaluminum compound, (B) a hydrocarbon diluent, (C) a heteroatomic ligand, and (D) a transition metal compound comprising Fe, Co, or Ni. The transition metal compound can be soluble in the hydrocarbon diluent.

A representative oligomerization process encompassed herein can comprise (i) contacting ethylene, an organic reaction medium, and a catalyst composition comprising (I) an organoaluminum compound, a heteroatomic ligand, and a transition metal compound comprising Fe, Co, or Ni, wherein the transition metal compound can be soluble in the organic reaction medium, or (II) an organoaluminum compound and a heteroatomic ligand transition metal compound complex comprising Fe, Co, or Ni, in an oligomerization reactor, (ii) forming an oligomer product in the oligomerization reactor, the oligomer product comprising hexenes and octenes, and (iii) discharging an effluent stream from the oligomerization reactor, the effluent stream comprising unreacted ethylene and the oligomer product.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description.

FIG. 1 presents a schematic diagram of an ethylene oligomerization process consistent with aspects of the invention.

FIG. 2 presents a ¹H NMR plot of Ligand 9.

FIG. 3 presents a ¹H NMR plot of Ligand 10.

FIG. 4 presents a ¹H NMR plot of Ligand 11.

FIG. 5 presents a ¹H NMR plot of Ligand 12.

FIG. 6 presents a ¹H NMR plot of Ligand 13.

FIG. 7 presents a ¹H NMR plot of Ligand 14.

FIG. 8 presents a ¹H NMR plot of Ligand 15.

FIG. 9 presents a ¹H NMR plot of Ligand 16.

FIG. 10 presents a ¹H NMR plot of Ligand 17.

FIG. 11 presents a ¹H NMR plot of Ligand 18.

FIG. 12 presents a ¹H NMR plot of Ligand 19.

FIG. 13 presents a ¹H NMR plot of Ligand 20.

FIG. 14 presents a ¹H NMR plot of Ligand 21.

FIG. 15 presents a ¹H NMR plot of Ligand 22.

FIG. 16 presents a ¹H NMR plot of Ligand 23, and FIG. 17 presents a zoomed-in view of the high chemical shift region of FIG. 16.

FIG. 18 presents a ¹H NMR plot of Ligand 24, and FIG. 19 presents a zoomed-in view of the high chemical shift region of FIG. 18.

FIG. 20 presents a ¹H NMR plot of Ligand 25, and FIG. 21 presents a zoomed-in view of the high chemical shift region of FIG. 20.

FIG. 22 presents a plot of catalyst activity versus catalyst storage time for the alkylaluminoxane composition and MMAO oligomerization experiments of Example 38.

FIG. 23 presents a plot of catalyst activity versus catalyst storage time for the alkylaluminoxane composition, alkylaluminoxane composition mixed with TIBA, and MMAO oligomerization experiments of Example 39.

FIG. 24 presents a plot of catalyst activity versus catalyst storage time for the alkylaluminoxane composition oligomerization experiments of Example 40.

While the inventions disclosed herein are susceptible to various modifications and alternative forms, only a few specific aspects have been shown by way of example in the drawings and described in detail below. The figures and detailed descriptions of these specific aspects are not intended to limit the breadth or scope of the inventive concepts or the appended claims in any manner. Rather, the figures and detailed descriptions are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present invention can comprise; alternatively, can consist essentially of; or alternatively, can consist of; a heteroatomic ligand, a transition metal compound, a hydrocarbon diluent, and an organoaluminum compound. The terms “a,” “an,” and “the” are intended to include plural alternatives, e.g., at least one, unless otherwise specified. For instance, the disclosure of “an organoaluminum compound” is meant to encompass one, or mixtures or combinations of two or more, organoaluminum compound(s), unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “oligomer” refers to a compound that contains from 2 to 20 monomer units. The terms “oligomerization product” and “oligomer product” include all products made by the “oligomerization” process, including the “oligomers” and products which are not “oligomers” (e.g., products which contain more than 20 monomer units, or solid polymer), but exclude other non-oligomer components of an oligomerization reactor effluent stream, such as unreacted ethylene, organic reaction medium, and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the active catalytic site, or the fate of the hydrocarbon diluent, the organoaluminum compound, and the heteroatomic ligand transition metal compound complex (or the hydrocarbon diluent, the organoaluminum compound, the heteroatomic ligand, and the transition metal compound) after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, when a chemical moiety having a certain number of carbon atoms is disclosed or claimed, the intent is to disclose or claim individually every possible number that such a range could encompass, consistent with the disclosure herein. For example, the disclosure that a moiety is a C₁ to C₁₈ hydrocarbyl group, or in alternative language, a hydrocarbyl group having from 1 to 18 carbon atoms, as used herein, refers to a moiety that can have 1, 2, 3, 4, 5, 6, 7, 8, 9, 10, 11, 12, 13, 14, 15, 16, 17, or 18 carbon atoms, as well as any range between these two numbers (for example, a C₁ to C₈ hydrocarbyl group), and also including any combination of ranges between these two numbers (for example, a C₂ to C₄ and a C₁₂ to C₁₆ hydrocarbyl group).

Similarly, another representative example follows for the molar ratio of Al:transition metal in the catalyst composition. By a disclosure that the molar ratio can range from 10:1 to 5,000:1, the intent is to recite that the molar ratio can be any ratio within the range and, for example, can include any range or combination of ranges from 10:1 to 5,000:1, such as from 50:1 to 3,000:1, from 75:1 to 3,000:1, from 75:1 to 2,000:1, from 100:1 to 2,000:1, or from 100:1 to 1,000:1, and so forth. Likewise, all other ranges disclosed herein should be interpreted in a manner similar to these examples.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Disclosed herein are heteroatomic ligand transition metal compound complexes as well as heteroatomic ligands and transition metal compounds, catalyst compositions containing the respective complexes (or the respective ligands and compounds), and ethylene oligomerization processes utilizing the catalyst compositions to produce 1-hexene and/or 1-octene.

An objective of this invention is to develop a catalyst system in which all the catalyst components are soluble in the process medium utilized in the oligomerization reactor. Another objective of this invention is to use an α-olefin as the process medium, wherein the α-olefin does not react (e.g., oligomerize) in the presence of the catalyst system. Another objective of this invention is to reduce or eliminate the use of alkane and aromatic diluents, leading to simplified separations downstream of the reactor. Yet another objective of this invention is to feed the respective catalyst components separately to the reactor, such that independent adjustment of the catalyst system (and the relative amounts of each component) can be accomplished in real-time. Still another objective of this invention is to develop catalyst systems that provide excellent catalyst activity in ethylene oligomerization processes, resulting in high yields of oligomer products, as well as being highly selective in the formation of desirable C₆-C₁₀ linear α-olefins.

A first catalyst composition consistent with aspects of this invention can contain (a) an organoaluminum compound, (b) a hydrocarbon diluent, and (c) a heteroatomic ligand transition metal compound complex comprising Fe, Co, or Ni. A second catalyst composition consistent with aspects of this invention can comprise (A) an organoaluminum compound, (B) a hydrocarbon diluent, (C) a heteroatomic ligand, and (D) a transition metal compound comprising Fe, Co, or Ni, and the transition metal compound can be soluble in the hydrocarbon diluent.

A representative oligomerization process consistent with aspects of this invention can comprise (i) contacting ethylene, an organic reaction medium, and a catalyst composition comprising (I) an organoaluminum compound, a heteroatomic ligand, and a transition metal compound comprising Fe, Co, or Ni, wherein the transition metal compound can be soluble in the organic reaction medium, or (II) an organoaluminum compound and a heteroatomic ligand transition metal compound complex comprising Fe, Co, or Ni, in an oligomerization reactor, (ii) forming an oligomer product in the oligomerization reactor, the oligomer product comprising hexenes and octenes, and (iii) discharging an effluent stream from the oligomerization reactor, the effluent stream comprising unreacted ethylene and the oligomer product. Optionally, in this oligomerization process, the catalyst composition in step (i) can further comprise a hydrocarbon diluent, and additionally or alternatively, hydrogen can be present in step (i) of the oligomerization process.

### Organoaluminum Compounds

Generally, the organoaluminum compound utilized in the catalyst compositions and oligomerization processes disclosed herein can be any organoaluminum compound which in conjunction with the heteroatomic ligand transition metal compound complex (or the transition metal compound and heteroatomic ligand) can catalyze the formation of an oligomer product. In an aspect, the organoaluminum compound can comprise, can consist essentially of, or can consist of, an aluminoxane, an alkylaluminum compound, or any combination thereof; alternatively, an aluminoxane; or alternatively, an alkylaluminum compound. In an aspect, the alkylaluminum compound can comprise, can consist essentially of, or can consist of, a trialkylaluminum, an alkylaluminum halide, an alkylaluminum alkoxide, or any combination thereof. In some aspects, the alkylaluminum compound can comprise, can consist essentially of, or can consist of, a trialkylaluminum, an alkylaluminum halide, or any combination thereof; alternatively, a trialkylaluminum, an alkylaluminum alkoxide, or any combination thereof; or alternatively, a trialkylaluminum. In other aspects, the alkylaluminum compound can be a trialkylaluminum; alternatively, an alkylaluminum halide; or alternatively, an alkylaluminum alkoxide. In an aspect, the aluminoxane utilized in the catalyst systems can comprise, can consist essentially of, or can consist of, any aluminoxane which in conjunction with the heteroatomic ligand transition metal compound complex (or the transition metal compound and heteroatomic ligand) can catalyze the formation of an oligomer product. In a non-limiting aspect, the aluminoxane can have a repeating unit characterized by Formula (III):

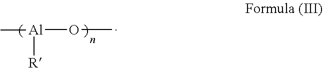

In formula (III), R′ is a linear or branched alkyl group. Alkyl groups of the aluminoxanes and alkylaluminum compounds are independently described herein and can be utilized without limitation to further describe the aluminoxanes having Formula (III) and/or the alkylaluminum compounds. Generally, n of Formula (III) can be greater than 1; or alternatively, greater than 2. In an aspect, n can range from 2 to 15; or alternatively, range from 3 to 10.

In an aspect, each halide of any alkylaluminum halide disclosed herein can independently be fluoride, chloride, bromide, or iodide; or alternatively, chloride, bromide, or iodide. In an aspect, each halide of any alkylaluminum halide disclosed herein can be fluoride; alternatively, chloride; alternatively, bromide; or alternatively, iodide.

In an aspect, each alkyl group of an aluminoxane and/or alkylaluminum compound independently can be a C₁ to C₂₀ alkyl group; alternatively, a C₁ to C₁₀ alkyl group; or alternatively, a C₁ to C₆ alkyl group. In an aspect, each alkyl group of an aluminoxane and/or alkylaluminum compound independently can be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some aspects, each alkyl group of an aluminoxane and/or alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

In an aspect, each alkoxide group of any alkylaluminum alkoxide disclosed herein independently can be a C₁ to C₂₀ alkoxy group, a C₁ to C₁₀ alkoxy group, or a C₁ to C₆ alkoxy group. In an aspect, each alkoxide group of any alkylaluminum alkoxide disclosed herein independently can be a methoxy group, an ethoxy group, a propoxy group, a butoxy group, a pentoxy group, a hexoxy group, a heptoxy group, or an octoxy group; alternatively, a methoxy group, an ethoxy group, a butoxy group, a hexoxy group, or an octoxy group. In some aspects, each alkoxide group of any alkylaluminum alkoxide disclosed herein independently can be a methoxy group, an ethoxy group, an n-propoxy group, an n-butoxy group, an iso-butoxy group, an n-hexoxy group, or an n-octoxy group; alternatively, a methoxy group, an ethoxy group, an n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, an n-hexoxy group; or alternatively, an n-octoxy group.

In a non-limiting aspect, useful trialkylaluminum compounds can include trimethylaluminum, triethylaluminum, tripropylaluminum, tributylaluminum, trihexylaluminum, trioctylaluminum, or mixtures thereof. In some non-limiting aspects, useful trialkylaluminum compounds can include trimethylaluminum, triethylaluminum, tripropylaluminum, tri-n-butylaluminum, tri-isobutylaluminum, trihexylaluminum, tri-n-octylaluminum, or mixtures thereof; alternatively, triethylaluminum, tri-n-butylaluminum, tri-isobutylaluminum, trihexylaluminum, tri-n-octylaluminum, or mixtures thereof; alternatively, triethylaluminum, tri-n-butylaluminum, trihexylaluminum, tri-n-octylaluminum, or mixtures thereof. In other non-limiting aspects, useful trialkylaluminum compounds can include trimethylaluminum; alternatively, triethylaluminum; alternatively, tripropylaluminum; alternatively, tri-n-butylaluminum; alternatively, tri-isobutylaluminum; alternatively, trihexylaluminum; or alternatively, tri-n-octylaluminum.

In a non-limiting aspect, useful alkylaluminum halides can include diethylaluminum chloride, diethylaluminum bromide, ethylaluminum dichloride, ethylaluminum sesquichloride, and mixtures thereof. In some non-limiting aspects, useful alkylaluminum halides can include diethylaluminum chloride, ethylaluminum dichloride, ethylaluminum sesquichloride, and mixtures thereof. In other non-limiting aspects, useful alkylaluminum halides can include diethylaluminum chloride; alternatively, diethylaluminum bromide; alternatively, ethylaluminum dichloride; or alternatively, ethylaluminum sesquichloride.

In a non-limiting aspect, the aluminoxane can comprise, consist essentially of, or consist of, methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or mixtures thereof. In some non-limiting aspects, the aluminoxane can comprise, consist essentially of, or consist of, methylaluminoxane (MAO), modified methylaluminoxane (MMAO), isobutyl aluminoxane, t-butyl aluminoxane, or mixtures thereof. In other non-limiting aspects, the aluminoxane can comprise, consist essentially of, or consist of, methylaluminoxane (MAO); alternatively, ethylaluminoxane; alternatively, modified methylaluminoxane (MMAO); alternatively, n-propylaluminoxane; alternatively, iso-propyl-aluminoxane; alternatively, n-butylaluminoxane; alternatively, sec-butylaluminoxane; alternatively, iso-butylaluminoxane; alternatively, t-butyl aluminoxane; alternatively, 1-pentyl-aluminoxane; alternatively, 2-pentylaluminoxane; alternatively, 3-pentyl-aluminoxane; alternatively, iso-pentyl-aluminoxane; or alternatively, neopentylaluminoxane.

Suitable aluminoxanes are not limited solely by those provided above. For example, the aluminoxane can comprise (or consist essentially of, or consist of) an alkylaluminoxane composition that comprises (i) an alkylaluminoxane having random repeating units of formula (A) and formula (B), and (ii) a hydrocarbon solvent, and the amount of aluminum in the composition can range from 0.1 to 20 wt. %. In formulas (A) and (B), R is methyl and R¹ is ethyl at a molar ratio of methyl:ethyl from 5:95 to 80:20:

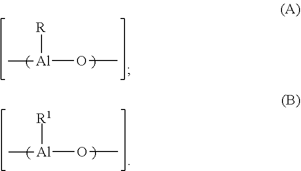

The alkylaluminoxane having random repeating units of formula (A) and formula (B) also encompasses structures that may have cross-linked or aggregated units resulting in non-linear 2D and 3D alkylaluminoxane structures including cluster/cage structures, such as described in Collins, Chem. Eur. J. 2021, 27, 15460-71, and references therein.

Suitable alkylaluminoxane compositions that can be used as the aluminoxane component of the catalyst composition can be produced by a process that comprises (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

Accordingly, a representative process for preparing an alkylaluminoxane composition can comprise (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

Generally, the features of these compositions and processes (e.g., the relative amount of TMA to TEA (or methyl to ethyl), the hydrocarbon solvent, the amount of aluminum in the composition, and the relative amount of water to aluminum, among others) are independently described herein and these features can be combined without limitation, and in any combination to further describe the disclosed compositions and processes. Moreover, additional steps can be performed before, during, and/or after the steps of the processes, and can be utilized without limitation and in any combination to further describe the processes for preparing alkylaluminoxane compositions, unless stated otherwise. Likewise, the alkylaluminoxane compositions can contain other materials or components, unless stated otherwise.

The alkylaluminoxane compositions—which are generally solutions of the alkylaluminoxane in the hydrocarbon solvent—can contain from 0.1 to 20 wt. % of aluminum. For example, the composition can contain from 1 to 20 wt. % aluminum in one aspect, from 2 to 15 wt. % aluminum in another aspect, from 3 to 12 wt. % aluminum in another aspect, from 3 to 7 wt. % aluminum in another aspect, from 4 to 12 wt. % aluminum in yet another aspect, and from 5 to 10 wt. % aluminum in still another aspect. These weight percentages are based on the weight of the aluminum (in any form) in the composition as compared to the total weight of the composition. The amount of aluminum in the alkylaluminoxane composition is determined by ICP analysis. Dilute alkylaluminoxane compositions also are contemplated herein, and such dilute compositions or solutions of the alkylaluminoxane in the hydrocarbon solvent can contain from 0.1 to 2 wt. % of aluminum, and more often, from 0.1 to 1.5 wt. %, from 0.1 to 1 wt. %, or from 0.2 to 0.8 wt. %, and the like, and the amount of aluminum can depend upon viscosity and pumping considerations during use of the alkylaluminoxane compositions.

It is believed that these alkylaluminoxane compositions, which are prepared from a mixture or combination of trimethylaluminum (TMA) and triethylaluminum (TEA) are more cost effective than traditional MAO and MMAO activators. Beneficially, these alkylaluminoxane composition are soluble in non-aromatic hydrocarbons, have excellent shelf-life and stability (in solution form), and have exceptional activating potential for certain transition metal catalysts, such as those employed in ethylene oligomerization processes.

The relative amounts of TMA:TEA or methyl:ethyl in the processes or alkylaluminoxane compositions are not particularly limited. Nonetheless, illustrative and non-limiting ranges include molar ratios of TMA:TEA (or molar ratios of methyl:ethyl) from 10:90 to 70:30, from 15:85 to 60:40, from 15:85 to 40:60, from 15:85 to 30:70, from 15:85 to 25:75, from 20:80 to 70:30, from 20:80 to 40:60, from 20:80 to 30:70, or from 20:80 to 25:75. Often, it can be beneficial for the amount of TEA (or ethyl) to be greater than that of TMA (or methyl). In such circumstances, the molar ratio of TMA:TEA (or the molar ratio of methyl:ethyl) can be from 15:85 to 40:60, from 15:85 to 30:70, from 15:85 to 25:75, from 20:80 to 40:60, from 20:80 to 30:70, or from 20:80 to 25:75, while not limited thereto.

The alkylaluminoxane compositions are generally substantially free of water (contain less than 1 wt. % water), due to the consumption of water in the process for producing the alkylaluminoxane composition. More often, these compositions contain less than 1000 ppm (by weight) water, less than 500 ppm water, or less than 100 ppm water. In addition, the alkylaluminoxane compositions also can contain TEA, or TMA, or both TEA and TMA—this is unreacted or free TEA and/or TMA that has not been consumed in the process for producing the alkylaluminoxane composition.

A variety of hydrocarbon solvents can be used in the alkylaluminoxane compositions and the processes for preparing the alkylaluminoxane compositions disclosed herein. For instance, the hydrocarbon solvent can comprise any suitable saturated aliphatic hydrocarbon, any suitable aromatic hydrocarbon, any suitable linear α-olefin, or any combination thereof.

The saturated aliphatic hydrocarbon can be a linear aliphatic hydrocarbon, a branched aliphatic hydrocarbon, or a cyclic aliphatic hydrocarbon, as well as combinations thereof. Thus, the hydrocarbon solvent can comprise a linear alkane, a branched alkane, a cyclic alkane, or a combination thereof. Illustrative examples of saturated aliphatic hydrocarbons that can be utilized as the solvent, either singly or in combination, include propane, butane (e.g., n-butane or isobutane), pentane (e.g., n-pentane, neopentane, cyclopentane, or isopentane), hexane, heptane, octane, cyclohexane, methyl cyclohexane, and the like, as well combinations thereof. In a particular aspect of this disclosure, the hydrocarbon solvent can comprise (or consist essentially of, or consist of) cyclohexane.

Additionally or alternatively, the hydrocarbon solvent can comprise an aromatic hydrocarbon, such as benzene, toluene, ethylbenzene, xylene, styrene, mesitylene, and the like. Combinations of two or more aromatic hydrocarbons can be utilized, if desired.

Illustrative examples of linear α-olefins that can be utilized as the hydrocarbon solvent, either singly or in combination, include 1-butene, 1-hexene, 1-octene, 1-decene, 1-dodecene, 1-tetradecene, and the like, as well as combinations thereof.

Generally, the alkylaluminoxane compositions described herein can contain at least 40 wt. % of the hydrocarbon solvent, and more often, at least 50 wt. %, at least 60 wt. %, at least 70 wt. %, at least 80 wt. %, or at least 85 wt. % of composition is the hydrocarbon solvent. These weight percentages are based on the weight of the hydrocarbon solvent(s) as compared to the total weight of the composition.

As disclosed herein, the alkylaluminoxane compositions can be a solution, such as at standard temperature and pressure (25° C. and 1 atm). This means that there is no visual precipitation (or minimal visual precipitation) of the alkylaluminoxane in the hydrocarbon solvent under standard conditions. Accordingly, the alkylaluminoxane composition can be a solution in an aromatic hydrocarbon solvent, such as toluene, or the alkylaluminoxane composition can be solution in a saturated aliphatic solvent, such as cyclohexane, or the alkylaluminoxane composition can be a solution in a linear α-olefin solvent, such as 1-hexene.

Beneficially, such alkylaluminoxane compositions are solutions at standard temperature and pressure (25° C. and 1 atm) that can be stable—no visual precipitation (or minimal visual precipitation) of the alkylaluminoxane in the hydrocarbon solvent—for at least 1 day, and in some aspects, for at least 3 days, for at least 7 days, for at least 10 days, or for at least 14 days.

Referring now to the processes for preparing alkylaluminoxane compositions and to alkylaluminoxane compositions produced by the processes, step (a) is directed to reacting trimethylaluminum (TMA), triethylaluminum (TEA), and water in a hydrocarbon solvent to form an alkylaluminoxane. The molar ratio options for TMA:TEA in step (a) are described hereinabove. The molar ratio of water:Al in step (a) can range from 0.2:1 to 1:1. More often, the molar ratio of water:Al can fall within the range of from 0.2:1 to 0.8:1, such as from 0.3:1 to 0.8:1, from 0.3:1 to 0.7:1, from 0.3:1 to 0.6:1, from 0.4:1 to 0.8:1, from 0.4:1 to 0.6:1, from 0.4:1 to 0.5:1, or from 0.5:1 to 0.6:1. While not limited thereto, molar ratios in the 0.3:1 to 0.8:1 or the 0.3:1 to 0.6:1 range, such as around 0.5:1, provide a good balance of catalytic activity and aluminum loss.

The order of addition of the components in step (a) is not particularly limited, but in one aspect, the TMA, TEA, and hydrocarbon solvent often can be combined first, followed by adding the water. In another aspect, TMA and the solvent can be combined first, followed by the water, and then TEA, while in yet another aspect, TEA and the solvent can be combined first, followed by the water, and then TMA.

Step (a) can be performed at any suitable temperature, but typically below the standard boiling point of the hydrocarbon solvent, and considering that the reaction in step (a) is exothermic. Representative temperature ranges include from 10° C. to 90° C., from 20° C. to 70° C., from 15° C. to 55° C., from 20° C. to 45° C., or from 20° C. to 30° C., although not limited thereto. In these and other aspects, these temperature ranges also are meant to encompass circumstances where step (a) is conducted at a series of different temperatures, instead of at a single fixed temperature, wherein at least one temperature falls within the respective ranges. The pressure at which step (a) is conducted is not particularly limited, but can be at an elevated pressure (e.g., from 5 psig to 100 psig), at atmospheric pressure, or at any suitable sub-atmospheric pressure. In some instances, step (a) is conducted at atmospheric pressure, eliminating the need for pressurized vessels and their associated cost and complexity. Step (a) can be performed for any suitable time period and the addition of water in step (a) can be take place over any suitable time period. Illustrative and non-limiting time periods (e.g., for the complete or slow addition of water) include a wide range of time periods, such as from 1 min to 10 hr, from 1 min to 6 hr, from 5 min to 6 hr, from 5 min to 2 hr, or from 15 min to 3 hr, but is not limited solely to these time periods. Other appropriate temperature, pressure, and time ranges are readily apparent from this disclosure.

It should be noted that a catalyst is not required in step (a) to form the alkylaluminoxane, and thus the alkylaluminoxane is generally formed in the substantial absence of a catalyst (i.e., less than 1 wt. % of a catalyst based on the total weight of the TMA, TEA, water, and hydrocarbon solvent in step (a)). For instance, there can be less than 1000 ppm (by weight) of the catalyst, less than 100 ppm of the catalyst, or less than 10 ppm of the catalyst, and more often, no catalyst is used as demonstrated by the examples that follow.

In step (b), insoluble aluminum-containing materials are removed from the solvent to form the alkylaluminoxane composition, which contains from 0.1 to 20 wt. % of aluminum. The step of removing the insoluble aluminum-containing materials from the solvent can include any suitable technique, e.g., draining, decanting, pressing, centrifuging, filtering, sedimenting, stripping, evaporating, drying, and the like, or any combination thereof, and the respective technique(s) can be performed once or more than once. Often, the insoluble aluminum-containing materials are removed from the solvent via filtration.

It is desired to minimize the amount of aluminum removed in step (b) based on total aluminum before step (b), such as to less than or equal to 40 wt. %, less than or equal to 30 wt. %, less than or equal to 20 wt. %, or less than or equal to 10 wt. %. However, the aluminum loss often must be balanced with the amount of water added, the molar ratio of TMA:TEA, and the resulting catalytic activity. Given these considerations, it is common for the amount of aluminum removed in step (b)—based on total aluminum before step (b)—to fall within a range from 10 to 50 wt. %, from 15 to 45 wt. %, from 5 to 30 wt. %, from 5 to 20 wt. %, or from 20 to 40 wt. %.

In aspects of this invention, the processes for preparing alkylaluminoxane compositions do not require a step of removing (e.g., via filtration) the insoluble aluminum-containing materials from the solvent to form the alkylaluminoxane composition. In these aspects, therefore, the process for preparing an alkylaluminoxane composition can comprise reacting trimethylaluminum (TMA), triethylaluminum (TEA), and water in a hydrocarbon solvent (any hydrocarbon solvent disclosed herein) to form the alkylaluminoxane composition (which contains insoluble aluminum-containing materials). The alkylaluminoxane composition contains from 0.1 to 20 wt. % of aluminum (or any amount of aluminum disclosed herein). The molar ratio of TMA:TEA is from 5:95 to 80:20 (or any molar ratio disclosed herein), and the molar ratio of water:Al is from 0.2:1 to 1:1 (or any molar ratio disclosed herein). While not limited thereto, the alkylaluminoxane composition can be prepared and then used (without removing the insoluble aluminum-containing materials) directly in any of the catalyst compositions and oligomerization processes disclosed herein.

### Hydrocarbon Diluents and Organic Reaction Mediums

The hydrocarbon diluent and the organic reaction medium used in the catalyst compositions and/or the oligomerization processes can be the same or different and independently can comprise any suitable saturated aliphatic hydrocarbon, any suitable aromatic hydrocarbon, any suitable linear α-olefin, or any combination thereof.

The saturated aliphatic hydrocarbon can be a linear aliphatic hydrocarbon, a branched aliphatic hydrocarbon, or a cyclic aliphatic hydrocarbon, as well as combinations thereof. Thus, the hydrocarbon diluent and/or the organic reaction medium can comprise a linear alkane, a branched alkane, a cyclic alkane, or a combination thereof. Illustrative examples of saturated aliphatic hydrocarbons that can be utilized as the hydrocarbon diluent (or the organic reaction medium, or both), either singly or in combination, include propane, butane (e.g., n-butane or isobutane), pentane (e.g., n-pentane, neopentane, cyclopentane, or isopentane), hexane, heptane, octane, cyclohexane, methyl cyclohexane, and the like, as well as combinations thereof. In a particular aspect of this disclosure, the hydrocarbon diluent and/or the organic reaction medium can comprise (or consist essentially of, or consist of) cyclohexane.

Additionally or alternatively, the hydrocarbon diluent and/or the organic reaction medium can comprise an aromatic hydrocarbon, such as benzene, toluene, ethylbenzene, xylene, styrene, mesitylene, and the like. Combinations of two or more aromatic hydrocarbons can be utilized, if desired.

Illustrative examples of linear α-olefins that can be utilized as the hydrocarbon diluent (or the organic reaction medium, or both), either singly or in combination, include 1-butene, 1-hexene, 1-octene, 1-decene, 1-dodecene, 1-tetradecene, and the like, as well as combinations thereof. In a particular aspect of this disclosure, the hydrocarbon diluent and the organic reaction medium are the same or different and independently comprise a linear α-olefin that comprises (or consists essentially of, or consists of) 1-butene; alternatively, 1-hexene; alternatively, 1-octene; alternatively, 1-decene; alternatively, 1-dodecene; alternatively, 1-tetradecene; or alternatively, any mixture or combination of linear α-olefins.

If the organoaluminum compound (or the aluminoxane) in the catalyst composition contains any of the alkylaluminoxane compositions disclosed herein, the resulting catalyst composition, therefore, also can contain a hydrocarbon solvent from the alkylaluminoxane composition, e.g., any suitable saturated aliphatic hydrocarbon or aromatic hydrocarbon. This hydrocarbon solvent can be the same as or different from the hydrocarbon diluent in the catalyst composition, and/or can be the same as or different from the organic reaction medium used in the oligomerization processes.

### Ligands and Metal Compounds and Complexes

Referring now to the heteroatomic ligand transition metal compound complex, any suitable heteroatomic ligand transition metal compound complex can be utilized in the catalyst compositions and the oligomerization processes provided herein, with the proviso that that the transition metal is Fe, Co, or Ni. Exemplary heteroatomic ligand transition metal compound complexes that are well suited for use in the disclosed catalyst compositions and oligomerization processes include those described, for example, in U.S. Pat. Nos. 6,534,691, 6,555,723, 6,683,187, 6,710,006, 7,037,988, 7,049,442, 7,053,020, 7,129,304, 7,179,871, 7,238,764, 7,268,096, 7,271,121, 7,304,159, 7,547,783, 7,589,245, 7,727,926, 7,728,160, 7,728,161, 7,977,269, 8,680,003, 8,865,610, 9,962,689, 10,407,360, 10,493,422, 10,464,862, 10,435,336, and 11,267,909.

Suitable heteroatomic ligand transition metal (Fe, Co, Ni) compound complexes are not limited solely by those provided above. For example, the heteroatomic ligand transition metal compound complex can have formula (IB) or formula (IIB). In the heteroatomic ligand transition metal compound complex having formula (IB), X is P or S; y is equal to 1 when X is S, and y is equal to 2 when X is P; M is Fe, Co, or Ni; m is an oxidation state of M; each Z independently is any suitable monoanionic ligand, such as H, a halogen, a C₁-C₁₈ hydrocarbyl group, or a C₁-C₁₈ halogenated hydrocarbyl group; R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and each R¹² independently is a C₁-C₁₈ hydrocarbyl group or a C₁-C₁₈ halogenated hydrocarbyl group. Optionally, two or more Z can be joined to form a dianionic or trianionic or polyanionic ligand to balance the oxidation state of M.

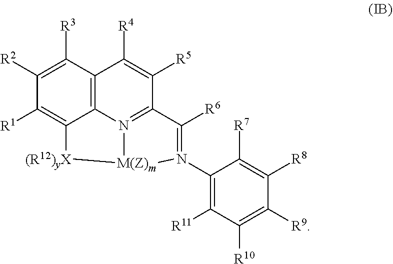

In the heteroatomic ligand transition metal compound complex having formula (IIB), X is P or S; y is equal to 1 when X is S, and y is equal to 2 when X is P; Y is O, NH, or CH₂; M is Fe, Co, or Ni; m is an oxidation state of M; each Z independently is any suitable monoanionic ligand, such as H, a halogen, a C₁-C₁₈ hydrocarbyl group, or a C₁-C₁₈ halogenated hydrocarbyl group; R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and each R^{A} independently is a C₁-C₁₈ hydrocarbyl group or a C₁-C₁₈ halogenated hydrocarbyl group. Optionally, two or more Z can be joined to form a dianionic or trianionic or polyanionic ligand to balance the oxidation state of M.

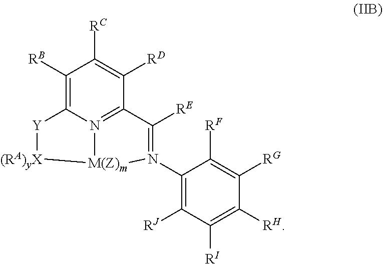

Referring now to the heteroatomic ligand, any suitable heteroatomic ligand can be utilized in the catalyst compositions and the oligomerization processes provided herein. Exemplary heteroatomic ligands that are well suited for use in the disclosed catalyst compositions and oligomerization processes include those described, for example, in U.S. Pat. Nos. 6,534,691, 6,555,723, 6,683,187, 6,710,006, 7,037,988, 7,049,442, 7,053,020, 7,129,304, 7,179,871, 7,238,764, 7,268,096, 7,271,121, 7,304,159, 7,547,783, 7,589,245, 7,727,926, 7,728,160, 7,728,161, 7,977,269, 8,680,003, 8,865,610, 9,962,689, 10,407,360, 10,493,422, 10,464,862, 10,435,336, and 11,267,909.

Suitable heteroatomic ligands are not limited solely by those provided above. For example, the heteroatomic ligand can have formula (IA) or formula (IIA). In the heteroatomic ligand having formula (IA), X is P or S; y is equal to 1 when X is S, and y is equal to 2 when X is P; R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and each R¹² independently is a C₁-C₁₈ hydrocarbyl group or a C₁-C₁₈ halogenated hydrocarbyl group.

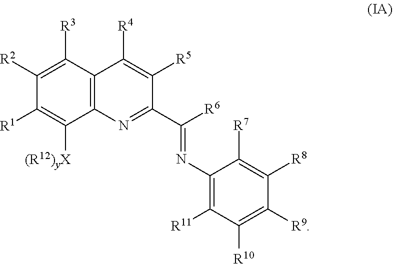

In the heteroatomic ligand transition metal compound complex having formula (IIA), X is P or S; y is equal to 1 when X is S, and y is equal to 2 when X is P; Y is O, NH, or CH₂; R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and each R^{A} independently is a C₁-C₁₈ hydrocarbyl group or a C₁-C₁₈ halogenated hydrocarbyl group.

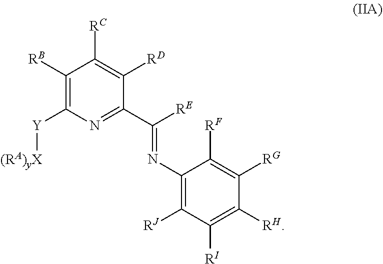

Unless otherwise specified, formulas (IA), (IB), (IIA), and (IIB) above, any other structural formulas disclosed herein, and any ligand, complex, compound, or species disclosed herein are not designed to show stereochemistry or isomeric positioning of the different moieties (e.g., these formulas are not intended to display rac or meso isomers, or R or S diastereoisomers), although such compounds are contemplated and encompassed by these formulas and/or structures, unless stated otherwise.

Referring first to formulas (IA) and (IB), in one aspect, X can be P and y can equal 2 in formulas (IA) and (IB), while in another aspect, X can be S and y can equal 1 in formulas (IA) and (IB). R¹ to R¹¹ independently can be H, a halogen, a nitro group, a C₁-C₁₈ hydrocarbyl group, or a C₁-C₁₈ halogenated hydrocarbyl group, and R⁵ and R⁶ can be joined to form a ring or ring system in formulas (IA) and (IB). It is contemplated that any of R¹ to R¹¹ can be either the same or different.

For example, R¹ to R¹¹ independently can be H, a halogen (e.g., F, Cl, Br), a nitro group, a C₁-C₁₈ hydrocarbyl group, or a C₁-C₁₈ halogenated hydrocarbyl group, and the hydrocarbyl and halogenated hydrocarbyl groups, independently, can be C₁-C₁₂ groups, or C₁-C₁₀ groups, C₁-C₈ groups, C₁-C₆ groups, or C₁-C₄ groups. Any hydrocarbyl group, independently, can be an alkyl group, a cycloalkyl group, an aryl group (e.g., a phenyl group or a naphthyl group, optionally substituted), or an aralkyl group (e.g., a benzyl group, optionally substituted), and likewise for halogenated hydrocarbyl groups.

In an aspect, R¹ to R¹¹ independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). For instance, R¹ to R⁶ independently can be H or a methyl group. Additionally or alternatively, R⁷ to R¹¹ independently can be, in some aspects, H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group (—NO₂), a pentafluorophenyl group, or a trifluoromethyl group (CF₃). Additionally or alternatively, R⁵ and R⁶ can be joined to form a ring or ring system. Likewise, in some aspects, R⁷ and R⁸ can be joined to form a ring or ring system.

Each R¹² in formulas (IA) and (IB) independently can be a C₁-C₁₈ hydrocarbyl group or a C₁-C₁₈ halogenated hydrocarbyl group, and any hydrocarbyl and halogenated hydrocarbyl options noted herein for R¹ to R¹¹ also can apply to R¹². Accordingly, in one aspect, each R¹² independently can be a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group (one or more methyl substituents), a naphthyl group, a benzyl group, a pentafluorophenyl group, or a trifluoromethyl group (CF₃). In another aspect, each R¹² independently can be a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

Referring now to the complex of formula (IB), M can be Fe, Co, or Ni; m can be an oxidation state of M; and each Z independently can be H, a halogen, a C₁-C₁₈ hydrocarbyl group, or a C₁-C₁₈ halogenated hydrocarbyl group. The metal in formula (IB), M, can be Fe, Co, or Ni, and thus in one aspect, for instance, M can be Fe or Ni, while in another aspect, M can be Fe; alternatively, M can be Co; or alternatively, M can be Ni.

Each Z in formula (IB) independently can be any suitable monoanionic ligand, such as H, a halogen, a C₁-C₁₈ hydrocarbyl group, or a C₁-C₁₈ halogenated hydrocarbyl group, and any halogen, hydrocarbyl, and halogenated hydrocarbyl options noted herein for R¹ to R¹¹ also can apply to Z. Accordingly, in one aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). In another aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a pentafluorophenyl group, or a trifluoromethyl group (CF₃). In yet another aspect, each Z can be Cl. In still another aspect, two or more Z can be joined to form a dianionic or trianionic or polyanionic ligand (e.g., catechol or a chelating group) to balance the oxidation state of M.

Referring now to formulas (IIA) and (IIB), in one aspect, X can be P and y can equal 2 in formulas (IIA) and (IIB), while in another aspect, X can be S and y can equal 1 in formulas (IIA) and (IIB). R^{B} to R^{J} independently can be H, a halogen, a nitro group, a C₁-C₁₈ hydrocarbyl group, or a C₁-C₁₈ halogenated hydrocarbyl group, and R^{D} and R^{E} can be joined to form a ring or ring system in formulas (IIA) and (IIB). It is contemplated that any of R^{B} to R^{J} can be either the same or different.

For example, R^{B} to R^{J} independently can be H, a halogen (e.g., F, Cl, Br), a nitro group, a C₁-C₁₈ hydrocarbyl group, or a C₁-C₁₈ halogenated hydrocarbyl group, and the hydrocarbyl and halogenated hydrocarbyl groups, independently, can be C₁-C₁₂ groups, or C₁-C₁₀ groups, C₁-C₈ groups, C₁-C₆ groups, or C₁-C₄ groups. Any hydrocarbyl group, independently, can be an alkyl group, a cycloalkyl group, an aryl group (e.g., a phenyl group or a naphthyl group, optionally substituted), or an aralkyl group (e.g., a benzyl group, optionally substituted), and likewise for halogenated hydrocarbyl groups.

In an aspect, R^{B} to R^{J} independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a nitro group, a pentafluorophenyl group, or a trifluoromethyl group (CF₃). For instance, R^{B} to R^{E} independently can be H or a methyl group. Additionally or alternatively, R^{F} to R^{J} independently can be, in some aspects, H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group (—NO₂), a pentafluorophenyl group, or a trifluoromethyl group (CF₃). Additionally or alternatively, R^{D} and R^{E} can be joined to form a ring or ring system. Likewise, in some aspects, R^{F} and R^{G} can be joined to form a ring or ring system.

Each R^{A} in formulas (IIA) and (IIB) independently can be a C₁-C₁₈ hydrocarbyl group or a C₁-C₁₈ halogenated hydrocarbyl group, and any hydrocarbyl and halogenated hydrocarbyl options noted herein for R^{B} to R^{J} also can apply to R^{A}. Accordingly, in one aspect, each R^{A} independently can be a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group (one or more methyl substituents), a naphthyl group, a benzyl group, a pentafluorophenyl group, or a trifluoromethyl group (CF₃). In another aspect, each R^{A} independently can be a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

In formulas (IIA) and (IIB), Y can be O, NH, or CH₂. Thus, in one aspect, Y is O, while in another aspect, Y is NH, and in yet another aspect, Y is CH₂.

Referring now to the complex of formula (IIB), M can be Fe, Co, or Ni; m can be an oxidation state of M; and each Z independently can be H, a halogen, a C₁-C₁₈ hydrocarbyl group, or a C₁-C₁₈ halogenated hydrocarbyl group. The metal in formula (IIB), M, can be Fe, Co, or Ni, and thus in one aspect, for instance, M can be Fe or Ni, while in another aspect, M can be Fe; alternatively, M can be Co; or alternatively, M can be Ni.

Each Z in formula (IIB) independently can be any suitable monoanionic ligand, such as H, a halogen, a C₁-C₁₈ hydrocarbyl group, or a C₁-C₁₈ halogenated hydrocarbyl group, and any halogen, hydrocarbyl, and halogenated hydrocarbyl options noted herein for R^{B} to R^{J} also can apply to Z. Accordingly, in one aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). In another aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a pentafluorophenyl group, or a trifluoromethyl group (CF₃). In yet another aspect, each Z can be Cl. In still another aspect, two or more Z can be joined to form a dianionic or trianionic or polyanionic ligand (e.g., catechol or a chelating group) to balance the oxidation state of M.

Generally, the transition metal compound or the transition metal compound of the heteroatomic ligand transition metal compound complexes described herein can have the formula M (X¹)ₚ. In this formula, M can be Fe, Co, or Ni, and p is an oxidation state of M. Each X¹ independently can be any suitable monoanionic ligand. Typically, the transition metal atom of the transition metal compound can have any positive oxidation state available to the transition metal atom. For instance, the cobalt atom and the nickel atom can have an oxidation state of +2, while the iron atom can have various oxidation states, including +2 and +3.

As disclosed herein, it can be beneficial for the transition metal compound to be soluble in the hydrocarbon diluent, in the organic reaction medium, or soluble in both the hydrocarbon diluent and the organic reaction medium. Solubility is considered at standard temperature and pressure (25° C. and 1 atm) and “soluble” means that there is no visual precipitation in a 0.001 wt. % solution. In some instances, the transition metal compound is soluble in a 0.004 wt. % solution, a 0.01 wt. % solution, a 0.1 wt. % solution, or a 1 wt. % solution in the hydrocarbon diluent (or the organic reaction medium) under standard conditions. Accordingly, the transition metal compound can form a solution in an aromatic hydrocarbon solvent, such as toluene, and/or can from a solution in a saturated aliphatic solvent, such as cyclohexane, and/or can form a solution in a linear α-olefin, such as 1-dodecene.

The monoanionic ligand (X¹) can be a halogen (e.g., fluorine or chlorine), a carboxylate, a β-diketonate, a hydrocarboxide, a nitrate, or a chlorate. The hydrocarboxide can be an alkoxide, an aryloxide, or an aralkoxide. Generally, any carboxylate of the transition metal compound independently can be a C₁ to C₂₀ carboxylate, or alternatively, a C₁ to C₁₀ carboxylate. In an aspect, each carboxylate independently can be acetate, a propionate, a butyrate, a pentanoate, a hexanoate, a heptanoate, an octanoate, a nonanoate, a decanoate, an undecanoate, or a dodecanoate; or alternatively, a pentanoate, a hexanoate, a heptanoate, an octanoate, a nonanoate, a decanoate, an undecanoate, or a dodecanoate. In some aspects, each carboxylate independently can be acetate, propionate, n-butyrate, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, capronate (n-hexanoate); alternatively, n-heptanoate; alternatively, caprylate (n-octanoate); or alternatively, 2-ethylhexanoate. In some aspects, the carboxylate can be triflate (trifluoroacetate). In other aspects, two or more X¹ can be joined to form a dianionic or trianionic or polyanionic ligand (e.g., catechol or a chelating group) to balance the oxidation state of M.

Generally, each β-diketonate of the transition metal compound independently can be any C₁ to C₂₀ a β-diketonate; or alternatively, any C₁ to C₁₀ β-diketonate. In an aspect, each β-diketonate independently can be acetylacetonate (i.e., 2,4-pentanedionate), hexafluoroacetylacetonate (i.e., 1,1,1,5,5,5-hexafluoro-2,4-pentanedionate), or benzoylacetonate; alternatively, acetylacetonate; alternatively, hexafluoroacetylacetonate; or alternatively, benzoylacetonate.

Generally, each hydrocarboxide of the transition metal compound independently can be any C₁ to C₂₀ hydrocarboxide; or alternatively, any C₁ to C₁₀ hydrocarboxide. In an aspect, each hydrocarboxide independently can be a C₁ to C₂₀ alkoxide; alternatively, a C₁ to C₁₀ alkoxide; alternatively, a C₆ to C₂₀ aryloxide; or alternatively, a C₆ to C₁₀ aryloxide. In an aspect, each alkoxide independently can be methoxide, ethoxide, a propoxide, or a butoxide; alternatively, methoxide, ethoxide, isopropoxide, or tert-butoxide; alternatively, methoxide; alternatively, an ethoxide; alternatively, an iso-propoxide; or alternatively, a tert-butoxide. In an aspect, the aryloxide can be phenoxide.

In some non-limiting aspects, the transition metal compound and/or the transition metal compound of the heteroatomic ligand transition metal compound complex can comprise, can consist essentially of, or consist of, a cobalt (II) halide, a nickel (II) halide, an iron (II) halide, a cobalt (II) carboxylate, a nickel (II) carboxylate, an iron (II) carboxylate, a cobalt (II) β-diketonate, a nickel (II) β-diketonate, or an iron (II) β-diketonate; alternatively, an iron (III) halide, an iron (III) carboxylate, or an iron (III) β-diketonate; alternatively, cobalt (II) nitrate, nickel (II) nitrate, iron (II) nitrate, or iron (III) nitrate; or alternatively, cobalt (II) acetylacetonate, nickel (II) acetylacetonate, iron (II) acetylacetonate, or iron (III) acetylacetonate.

While not shown in the transition metal compound names and formulas and/or heteroatomic ligand transition metal compound complex formulas and structures provided herein, one of ordinary skill in the art will recognize that a neutral ligand, Q, can be associated with the transition metal compounds and/or the heteroatomic ligand transition metal compound complexes described/depicted herein which do not explicitly disclose/depict a neutral ligand. Consequently, transition metal compounds and/or heteroatomic ligand transition metal compound complexes having a neutral ligand, Q, can be considered as equivalent to the transition metal compounds and/or heteroatomic ligand transition metal compound complexes depicted herein not having the neutral ligand, Q. Additionally, it should be understood that while some of the transition metal compounds and/or heteroatomic ligand transition metal compound complexes described/depicted/provided herein do not formally show the presence of a neutral ligand, the transition metal compounds and/or heteroatomic ligand transition metal compound complexes having neutral ligands (e.g., nitriles and ethers, among others) are fully contemplated and encompassed herein as potential transition metal compounds and/or heteroatomic ligand transition metal compound complexes that can be utilized in the catalyst systems and ethylene oligomerization processes of the present disclosure.

Generally, the neutral ligand of any transition metal compound and/or heteroatomic ligand transition metal compound complex, when present, independently can be any neutral ligand that forms an isolatable compound with the transition metal compound and/or heteroatomic ligand transition metal compound complex. In an aspect, each neutral ligand independently can be a nitrile or an ether; alternatively, a nitrile; or alternatively, an ether. The number of neutral ligands, q, can be any number that forms an isolatable compound with the transition metal compound, and/or heteroatomic ligand transition metal compound complex. In an aspect, the number of neutral ligands can be from 0 to 6; alternatively, 0 to 3; alternatively, 0; alternatively, 1; alternatively, 2; alternatively, 3; or alternatively, 4.

Generally, each nitrile ligand independently can be a C₂ to C₂₀ nitrile; or alternatively, a C₂ to C₁₀ nitrile. In an aspect, each nitrile independently can be acetonitrile, propionitrile, a butyronitrile, benzonitrile, or any combination thereof; alternatively, acetonitrile; alternatively, propionitrile; alternatively, a butyronitrile; or alternatively, benzonitrile.

Generally, each ether ligand independently can be a C₂ to C₄₀ ether; alternatively, a C₂ to C₃₀ ether; or alternatively, a C₂ to C₂₀ ether. In some aspects, each ether ligand independently can be dimethyl ether, diethyl ether, a dipropyl ether, a dibutyl ether, methyl ethyl ether, a methyl propyl ether, a methyl butyl ether, tetrahydrofuran, a dihydrofuran, 1,3-dioxolane, tetrahydropyran, a dihydropyran, a pyran, a dioxane, furan, benzofuran, isobenzofuran, dibenzofuran, diphenyl ether, a ditolyl ether, or any combination thereof; alternatively, dimethyl ether, diethyl ether, a dipropyl ether, a dibutyl ether, methyl ethyl ether, a methyl propyl ether, a methyl butyl ether, or any combination thereof; tetrahydrofuran, a dihydrofuran, 1,3-dioxolane, tetrahydropyran, a dihydropyran, a pyran, a dioxane, or any combination thereof; furan, benzofuran, isobenzofuran, dibenzofuran, or any combination thereof; diphenyl ether, a ditolyl ether, or any combination thereof; alternatively, dimethyl ether; alternatively, diethyl ether; alternatively, a dipropyl ether; alternatively, a dibutyl ether; alternatively, methyl ethyl ether; alternatively, a methyl propyl ether; alternatively, a methyl butyl ether; alternatively, tetrahydrofuran; alternatively, a dihydrofuran; alternatively, 1,3-dioxolane; alternatively, tetrahydropyran; alternatively, a dihydropyran; alternatively, a pyran; alternatively, a dioxane; alternatively, furan; alternatively, benzofuran; alternatively, isobenzofuran; alternatively, dibenzofuran; alternatively, diphenyl ether; or alternatively, a ditolyl ether.

While the heteroatomic ligand transition metal compound complex formulas and structures provided herein are shown as neutral complexes, one of ordinary skill in the art will recognize that heteroatomic ligand transition metal compound complexes can comprise or can exist as “ate” complexes comprising a negatively charged heteroatomic ligand transition metal compound complex and an associated positively charged metal or metal complex cation. Additionally, it should be understood that while the heteroatomic ligand transition metal compound complexes described/depicted/provided herein are shown as neutral complexes, the “ate” complexes comprising a negatively charged heteroatomic ligand transition metal compound complex and an associated positively charged metal or metal complex cation are implicitly and fully contemplated as potential heteroatomic ligand transition metal compound complexes that can be utilized in the catalyst systems and ethylene oligomerization processes of the present disclosure.

### Catalyst Compositions and Oligomerization Processes

In the catalyst composition, the relative amount of the organoaluminum compound versus that of the heteroatomic ligand transition metal compound complex (or versus that of the heteroatomic ligand and the transition metal compound) is not particularly limited. Nonetheless, molar ratios of Al:transition metal (for example, Al:Co or Al:Fe) or Al:ligand in the catalyst compositions and oligomerization processes disclosed herein can range from 10:1 to 5,000:1, such as from 50:1 to 3,000:1, from 75:1 to 3,000:1, from 75:1 to 2,000:1, from 100:1 to 2,000:1, or from 100:1 to 1,000:1, and the like. If, for example, more than one transition metal (or ligand) and/or more than one organoaluminum are employed, these ratios are based on the total moles of respective transition metals, ligands, and organoaluminums.

When both a heteroatomic ligand and a transition metal compound are present, the molar ratio of ligand:transition metal often ranges from 20:1 to 1:20, and more often, from 10:1 to 1:10, from 8:1 to 1:8, from 5:1 to 1:5, from 4:1 to 1:4, or from 2:1 to 1:2, and the like. In some aspects, the transition metal compound is present in a molar excess relative to the heteroatomic ligand, although this is not a requirement.

In an aspect, the heteroatomic ligand (compound) utilized in the catalyst composition and oligomerization process can be Ligand 5, Ligand 7, Ligand 9, or Ligand 11, or a mixture or combination thereof, as shown below in Table 1. Thus, for example, the heteroatomic ligand (compound) utilized in the catalyst composition and oligomerization process can be Ligand 5; alternatively, Ligand 7; alternatively, Ligand 9; or alternatively, Ligand 11.

In another aspect, the heteroatomic ligand (compound) utilized in the catalyst composition and oligomerization process can be Ligand 6, Ligand 8, Ligand 10, Ligand 12, Ligand 13, or Ligand 15, or a mixture or combination thereof, as shown below in Table 1. Thus, for example, the heteroatomic ligand (compound) utilized in the catalyst composition and oligomerization process can be Ligand 6; alternatively, Ligand 8; alternatively, Ligand 10; alternatively, Ligand 12; alternatively, Ligand 13; or alternatively, Ligand 15.

The components of the catalyst composition can be combined at any suitable temperature, such as from 0° C. to 90° C., from 20° C. to 70° C., from 15° C. to 55° C., from 20° C. to 45° C., or from 20° C. to 30° C. (room temperature can be conveniently used), although not limited thereto. The catalyst composition can be formed in the presence or absence of an olefin (e.g., the olefin to be oligomerized, such as ethylene). If the catalyst composition is formed in a reactor at the time of contacting the olefin, then the appropriate pressures and temperatures will be those that are typical of the oligomerization process, discussed further below.

Beneficially, catalyst compositions containing any of the alkylaluminoxane compositions disclosed herein are stable at standard temperature and pressure (25° C. and 1 atm). In this regard, the relationship between catalyst age and productivity is generally constant for at least one day, at least 2 days, at least 3 days, or at least 5 days. For instance, the change in productivity over time (A productivity/A time) can be less than or equal to 20% in one day, or less or equal to 15% in one day, or less than or equal to 10% in one day. The A productivity is the productivity at time zero minus the productivity at the particular time interval (e.g., 1 day). The catalyst composition stability is demonstrated in the Examples that follow.

When the organoaluminum component of the catalyst composition is any of the alkylaluminoxane compositions disclosed herein, an illustrative process for producing the catalyst composition can comprise performing any of the processes to produce the alkylaluminoxane composition disclosed herein, and then contacting the alkylaluminoxane composition with a heteroatomic ligand transition metal compound complex (or a heteroatomic ligand and a transition metal compound), and a hydrocarbon diluent to form the catalyst composition. As discussed herein, this can occur prior to the introduction of the catalyst composition into an oligomerization reactor, or the catalyst composition can be formed within the oligomerization reactor.

Referring now particularly to oligomerization processes, in step (i) ethylene, an organic reaction medium, and a catalyst composition comprising (I) an organoaluminum compound, a heteroatomic ligand, and a transition metal compound comprising Fe, Co, or Ni, wherein the transition metal compound can be soluble in the organic reaction medium, or (II) an organoaluminum compound and a heteroatomic ligand transition metal compound complex comprising Fe, Co, or Ni, are contacted in an oligomerization reactor. Ethylene, the organic reaction medium, and the catalyst composition (and hydrogen, if used) can be combined in any order or sequence and introduced into the oligomerization reactor separately or in any combination. For example, hydrogen and ethylene can be combined and fed to the reactor separately from the catalyst composition. This invention is not limited by the manner in which the respective feed streams are introduced into the reactor.

In one aspect, for instance, the catalyst composition can be formed first and then introduced into the oligomerization reactor. In this aspect, the organoaluminum compound is contacted with the heteroatomic ligand transition metal compound complex, or with the heteroatomic ligand and the transition metal compound, prior to being introduced into the reactor. In another aspect, however, the catalyst composition can be formed within the oligomerization reactor. In this aspect, the organoaluminum compound and the heteroatomic ligand transition metal compound complex (or at least one of the heteroatomic ligand and the transition metal compound) are introduced separately into the reactor, and the catalyst composition is formed within the reactor.

In some aspects, the organoaluminum compound, the heteroatomic ligand, and the transition metal compound are introduced separately into the oligomerization reactor, while in other aspects, at least a portion of the heteroatomic ligand and at least a portion the transition metal compound are combined to form at least a portion of the heteroatomic ligand transition metal compound complex before being introduced into the oligomerization reactor. In any of these above-described options, the organic reaction medium and the organoaluminum compound can combined and then introduced into the oligomerization reactor. Moreover, ethylene can be—and often is—introduced into the reactor separately from the catalyst composition (or catalyst system components).

In an aspect, after initial reactor start-up, no organic reaction medium is fed to the oligomerization reactor. Effectively, the oligomer product formed in step (ii)—discussed further below—acts as the organic reaction medium. In this aspect, the catalyst composition is highly selective for ethylene oligomerization and does not react appreciably with the hexenes, octenes, and other oligomers that are present in the oligomer product.

Referring now to step (ii), an oligomer product is formed in the oligomerization reactor, and the oligomer product comprises hexenes and octenes (amongst other oligomers). The oligomerization reactor in which the oligomer product is formed in step (ii) (and in which the components in step (i) are contacted) can comprise any suitable reactor. Non-limiting examples of reactor types can include a stirred tank reactor, a plug flow reactor, or any combination thereof; alternatively, a fixed bed reactor, a continuous stirred tank reactor, a loop reactor, a solution reactor, a tubular reactor, a recycle reactor, or any combination thereof. For instance, in one aspect, the oligomerization reactor can comprise a continuous stirred tank reactor, while in another aspect, the oligomerization reactor can comprise a loop reactor. In some aspects, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

Forming the oligomer product in the oligomerization reactor can be accomplished at any suitable oligomerization temperature and pressure. Often, the oligomer product can be formed at a minimum temperature of 0° C., 20° C., 30° C., 40° C., 45° C., or 50° C.; additionally or alternatively, at a maximum temperature of 165° C., 160° C., 150° C., 140° C., 130° C., 115° C., 100° C., or 90° C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 20 to 160, from 20 to 115, from 40 to 160, from 40 to 140, from 50 to 150, from 50 to 140, from 50 to 130, from 50 to 100, from 60 to 115, from 70 to 100, or from 75 to 95° C. Other appropriate oligomerization temperatures and temperature ranges are readily apparent from this disclosure.

The oligomer product can be formed at a minimum pressure (or ethylene partial pressure) of 50 psig (344 kPa), 100 psig (689 kPa), 200 psig (1.4 MPa), or 250 psig (1.5 MPa); additionally or alternatively, at a maximum pressure (or ethylene partial pressure) of 4,000 psig (27.6 MPa), 3,000 psig (20.9 MPa), 2,000 psig (13.8 MPa), or 1,500 psig (10.3 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 50 psig (344 kPa) to 4,000 psig (27.6 MPa), from 100 psig (689 kPa) to 3,000 psig (20.9 MPa), from 100 psig (689 kPa) to 2,000 psig (13.8 MPa), from 200 psig (1.4 MPa) to 2,000 psig (13.8 MPa), from 200 psig (1.4 MPa) to 1,500 psig (10.3 MPa), or from 250 psig (1.5 MPa) to 1,500 psig (10.3 MPa). Other appropriate oligomerization pressures (or ethylene partial pressures) are readily apparent from this disclosure.

When used, hydrogen can be fed directly to the reactor, or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

In step (iii) of the ethylene oligomerization process, an effluent stream is discharged from the oligomerization reactor, and the effluent stream contains unreacted ethylene and the oligomer product. The amount of conversion of ethylene in the oligomerization reactor is not particularly limited, and generally the minimum ethylene conversion can be at least 20, 30, 35, 40, 45, or 50 wt. %, while the maximum ethylene conversion can be 99, 95, 90, 80, 75, 70, or 65 wt. %. Generally, the ethylene conversion in the reactor can range from any minimum conversion to any maximum conversion described herein. For instance, the ethylene conversion can range from 20 to 95 wt. %, from 30 to 90 wt. %, from 40 to 80 wt. %, from 50 to 70 wt. %, or from 55 to 65 wt. %. The ethylene conversion is based on the amount of ethylene entering the reactor and the amount of (unreacted) ethylene in the effluent stream.

Among other constituents, the effluent stream contains the oligomer product, which can comprise hexenes and octenes, as well as other C₄⁺ linear alpha olefins. The amount of octenes in the oligomer product typically can fall within a range from 5 to 99 wt. %, based on the total amount of oligomers in the oligomer product. In an aspect, the minimum amount of octenes in the oligomer product can be 5, 10, 20, 30 or 40 wt. %. In another aspect, the maximum amount of octenes in the oligomer product can be 99, 95, 92.5, 90, 87.5, or 85 wt. %. Generally, the amount of octenes in the oligomer product can range from any minimum amount of octenes in the oligomer product to any maximum amount of octenes in the oligomer product described herein. For instance, the amount of octenes—based on the total weight of oligomers in the oligomer product—can be from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes.

Additionally or alternatively, the oligomer product can contain any suitable amount of hexenes. In an aspect, the minimum amount of hexenes in the oligomer product can be 10, 15, 20, 25, 30, or 35 wt. %. In another aspect, the maximum amount of hexenes in the oligomer product can be 75, 65, 60, 55, or 50 wt. %. Generally, the amount of hexenes in the oligomer product can range from any minimum amount of hexenes in the oligomer product to any maximum amount of hexenes in the oligomer product described herein. For instance, the amount of hexenes—based on the total weight of oligomers in the oligomer product—can be from 10 to 75 wt. %, from 15 to 65 wt. %, from 20 to 60 wt. %, from 25 to 55 wt. %, or from 30 to 50 wt. % hexenes.

Optionally, the disclosed oligomerization processes can further comprise a step of contacting the effluent stream—after discharging from the oligomerization reactor—with a catalyst system deactivating agent. While not limited thereto, the catalyst system deactivating agent can be a C₄-C₁₈ alcohol, and the term alcohol is used generically to include mono-ols, diols, and polyols, therefore the catalyst system deactivating agent can comprise a mono alcohol compound, a diol compound, a polyol compound, or any combination thereof.

Consistent with particular aspects of this invention, the catalyst system deactivating agent can comprise a butanol, a pentanol, a hexanol, a heptanol, an octanol, a nonanol, a decanol, an undecanol, and the like, as well as any mixture or combination thereof. Specific examples of catalyst system deactivating agents that can be utilized herein include, for instance, 1-butanol, 2-butanol, iso-butanol, sec-butanol, t-butanol, 1-pentanol, cyclopentanol, 1-hexanol, 2-hexanol, 3-hexanol, cyclohexanol, 1-heptanol, 2-heptanol, 3-heptanol, 4-heptanol, 1-octanol, 2-octanol, 3-octanol, 4-octanol, 2-ethyl-1-hexanol, 2-methyl-3-heptanol, 1-nonanol, 1-decanol, 2-decanol, 3-decanol, 4-decanol, 5-decanol, 1-undecanol, 2-undecanol, 7-methyl-2-decanol, a 1-docecanol, a 2-dodecanol, 2-ethyl-1-decanol, and the like, as well any mixture or combination thereof. In a particular aspect disclosed herein, the catalyst system deactivating agent can comprise 2-ethyl-hexanol.

Optionally, the disclosed oligomerization processes—after step (iii)—can further comprise a step of separating (or isolating) a C₆ stream and/or a step of separating (or isolating) a C₈ stream from the oligomer product. The separated (or isolated) C₆ stream can contain any suitable amount of hexene(s), such as at least 96, at least 97, at least 98, or at least 99 wt. % hexene(s), based on the total weight of the C₆ stream, and the separated (or isolated) C₈ stream can contain any suitable amount of octene(s), such as at least 96, at least 97, at least 98, or at least 99 wt. % octene(s), based on the total weight of the C₈ stream.

Generally, a vast majority of these C₆ and C₈ streams are the desirable α-olefin products, 1-hexene and 1-octene, respectively. While not limited thereto, the C₆ stream can contain, for example, at least 90 wt. % 1-hexene, and more often at least 92.5 wt. %, at least 95 wt. %, at least 97.5 wt. %, at least 98 wt. %, at least 98.5 wt. %, or at least 99 wt. % 1-hexene, based on the total weight of the hexene(s) in the C₆ stream. Likewise, the C₈ stream can contain, for example, at least 95 wt. % 1-octene, and more often, at least 96 wt. %, at least 96.5 wt. %, at least 97 wt. %, at least 97.5 wt. %, at least 98 wt. %, or 98.5 wt. % 1-octene, based on the total weight of the octene(s) in the Ca stream.

An oligomerization process consistent with aspects of this invention can comprise (1) performing any of the processes to produce the alkylaluminoxane composition disclosed herein, (2) contacting ethylene, a catalyst composition, an organic reaction medium, and optionally hydrogen, in an oligomerization reactor, (3) forming an oligomer product in the oligomerization reactor, the oligomer product comprising hexenes and octenes, and (4) discharging an effluent stream from the oligomerization reactor, the effluent stream comprising unreacted ethylene and the oligomer product. The catalyst composition can comprise the alkylaluminoxane composition, a hydrocarbon diluent, a heteroatomic ligand, and a transition metal compound comprising Fe, Co, or Ni (or a heteroatomic ligand transition metal compound complex). The transition metal compound can be soluble in the hydrocarbon diluent, in the organic reaction medium, or both. The hydrocarbon diluent can be the same as or different from the organic reaction medium. As discussed herein, the catalyst composition can be formed prior to the introduction of the catalyst composition into an oligomerization reactor, or the catalyst composition can be formed within the oligomerization reactor.

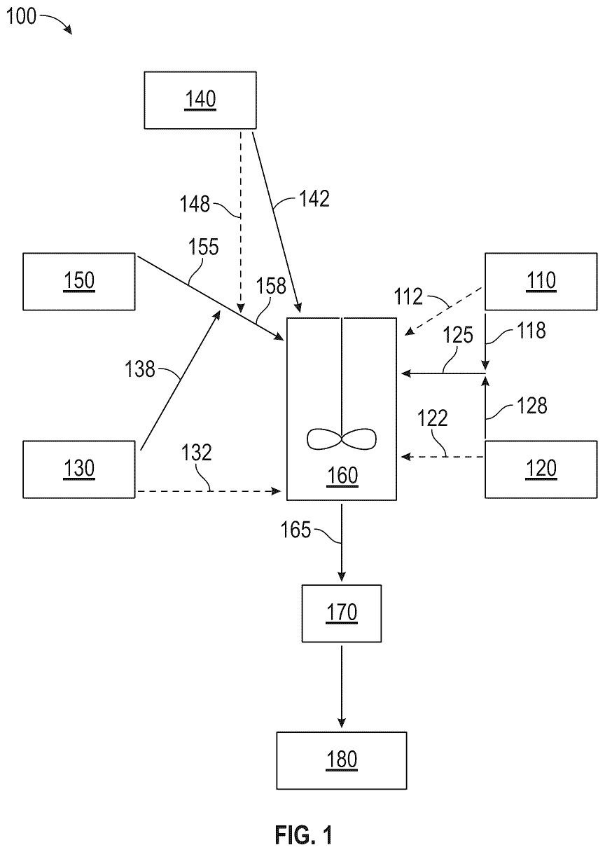

Referring now to FIG. 1, which illustrates a representative and non-limiting ethylene oligomerization process 100 consistent with aspects of the invention. In FIG. 1, ethylene 140 is fed separately/directly 142, an activator 130 (organoaluminum compound) is fed separately/directly 132, and an organic reaction medium 150 is fed 155 to an ethylene oligomerization reactor 160, which can be a loop reactor or a CSTR, although not limited thereto. Ethylene 140 can be fed separately/directly 142 to the reactor 160 or combined 148 with the organic reaction medium and then the resulting mixture is fed 158 to reactor 160. Likewise, the activator 130 can be fed separately/directly 132 to the reactor 160 or combined 138 with the organic reaction medium and then the resulting mixture is fed 158 to reactor 160.

Other components of the catalyst system, such as a heteroatomic ligand 110 and a Fe source 120 (transition metal compound) can be fed separately/directly 112, 122 into reactor 160. Thus, the catalyst system can be formed within the oligomerization reactor 160. A suitable hydrocarbon diluent can be used to feed 112, 122 the ligand and the Fe source into reactor 160, and the hydrocarbon diluent can be the same as or different from organic reaction medium 150. In another option illustrated in FIG. 1, some or all of the heteroatomic ligand and some or all of the transition metal compound are combined 118, 128, thereby forming a heteroatomic ligand transition metal compound complex, which is then fed 125 into oligomerization reactor 160.

Thus, for example, ethylene can be fed directly to the reactor in FIG. 1, and the activator can be combined with the organic reaction medium and introduced together into the reactor. The heteroatomic ligand and the transition metal compound (Fe source) also can be combined at a suitable relative amount and introduced together into the reactor.

An effluent stream 165 is discharged from the reactor in FIG. 1, and the effluent stream contains unreacted ethylene, an oligomer product comprising hexenes and octenes, and residual catalyst, among other constituents. The effluent stream is quenched 170 by contacting with a catalyst system deactivating agent, and the effluent stream and oligomer product are then subjected to a fractionation scheme 180, including a series of separation and isolation steps that result in, for instance, a C₆ stream containing predominantly 1-hexene and a C₈ stream containing predominantly 1-octene.

In one aspect consistent with FIG. 1, a tridentate ligand is dissolved in a hydrocarbon diluent and combined with a soluble Fe source either prior to or in the reactor, whereupon subsequent contacting or combining with an aluminoxane activator generates the active catalyst composition. Ethylene is oligomerized in a continuous reactor in single phase operation mode at a temperature of at least 75° C. to produce C₄⁺ linear α-olefins. Suitable organic reaction mediums in the reactor include 1-dodecene, 1-tetradecene, cyclohexane, or simply the resulting oligomer product or reactor effluent stream.

In another aspect consistent with FIG. 1, a diaryl phosphine ligand (e.g., the phosphine aryl groups have no ortho substituents, and the aryl-imino group has only one ortho substituent) is dissolved in a hydrocarbon diluent and combined with iron (III) octanoate either prior to or in the reactor, whereupon subsequent contacting or combining with an alkylaluminoxane composition generates the active catalyst composition. Ethylene is oligomerized in a continuous loop reactor in single phase operation at a temperature of at least 75° C. to produce C₄⁺ linear α-olefins. The organic reaction medium is 1-dodecene or simply the resulting oligomer product or reactor effluent stream.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

Disclosed below are methods for preparing heteroatomic ligands that are utilized in the preparation of the heteroatomic ligand transition metal compound complexes and catalyst compositions of Examples 1-21. The heteroatomic ligand transition metal compound complexes are prepared by combining the heteroatomic ligands with a transition metal compound (e.g., a soluble iron source). Advantageously, the inclusion of the disclosed heteroatomic ligand transition metal compound complexes in catalyst compositions leads to catalyst systems that exhibit high catalyst productivities and C₄-C₂₆ oligomer product selectivity. Additionally, the preparation methods of intermediate compounds necessary for the synthesis of the heteroatomic ligands are disclosed below.

For ¹H NMR data and ³¹P NMR data, the general procedure utilized a Bruker Instrument (e.g., Av400x) and approximately 3-10 mg of the respective compound dissolved in an appropriate NMR solvent (e.g., C₆D₆ and/or CDCl₃).

### Synthesis of Intermediate Compounds

Intermediate C (1-(6-aminopyridin-2-yl)ethan-1-one) was prepared as follows. A solution of 6-aminopicolinonitrile was prepared (1 g, 8.4 mmol) in THF (20 mL), stirred at 0° C. for 15 min, and MeMgBr (14 mL, 42.0 mmol) was added dropwise. The reaction was stirred for 4 hr at 25° C. and then quenched with NH₄Cl and extracted with EtOAc. The solution was rinsed with brine and dried with Na₂SO₄. The volatiles were removed in vacuo. The Intermediate C compound was obtained as a brown solid which turned yellow upon scraping with a spatula (1.12 g, 96% yield). ¹H NMR (500 MHZ, CDCl₃), δ=7.56 (t, J=7.8, 1H), 7.4 (d, J=7.4, 1H), 6.67 (d, J=8.2, 1H), 4.53 (brs, 2H), 2.63 (s, 3H). The chemical structure of Intermediate C is shown below:

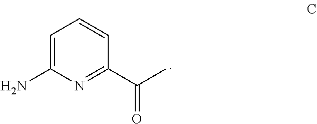

Intermediate D (6-(1-(mesitylimino)ethyl) pyridin-2-amine) was prepared as follows. In a round bottom flask, a solution of 1-(6-aminopyridin-2-yl) ethan-1-one (1 g, 7.35 mmol) was added to 2,4,6-trimethylaniline (1.55 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (1:1 hexane to EtOAc with 1% NEt₃) to yield the Intermediate D compound (0.5505 g, 31.8% yield). ¹H NMR (500 MHz, CDCl₃) δ=7.66 (d, J=7.5, 1H), 7.54 (t, J=7.8, 1H), 6.86 (s, 2H), 6.58 (d, J=7.7, 1H), 4.46 (brs, 2H), 2.28 (s, 3H), 2.08 (s, 3H), 1.99 (s, 6H). The chemical structure of Intermediate D is shown below:

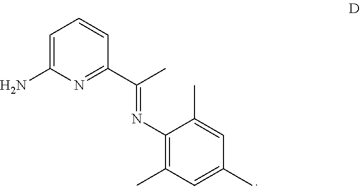 [SMILES: C/C(=N\c1c(C)cc(C)cc1C)c1cccc(N)n1]

Intermediate E (6-(1-((2,4-dimethylphenyl)imino) ethyl) pyridin-2-amine) was prepared as follows. In a round bottom flask, a solution of 1-(6-aminopyridin-2-yl) ethan-1-one (1 g, 7.35 mmol) was added to 2,4-dimethylaniline (1.35 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (5:1 hexane to EtOAc with 1% NEt₃) to yield the Intermediate E compound (117 mg, 6.6% yield). ¹H NMR (500 MHz, CDCl₃) δ=7.62-7.60 (d, J=7.53, 1H), 7.55-7.52 (t, J=7.92, 1H), 7.02 (s, 1H), 6.59-6.57 (d, J=8.15, 1H), 6.56-6.54 (d, J=7.91, 1H), 4.45 (s, 2H), 2.31 (s, 3H), 2.18 (s, 3H), 2.06 (s, 3H). The chemical structure of Intermediate E is shown below:

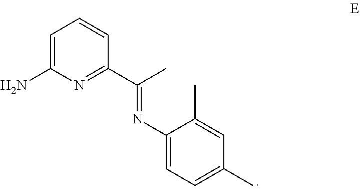 [SMILES: C/C(=N\c1ccc(C)cc1C)c1cccc(N)n1]

Intermediate F (6-(1-((2,4-dimethylphenyl)imino) ethyl) pyridin-2-amine) was prepared as follows. In a round bottom flask, a solution of 1-(6-aminopyridin-2-yl) ethan-1-one (1 g, 7.35 mmol) was added to 2,6-methylaniline (1.35 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo and the residues were purified via flash chromatography (1:1 hexane to EtOAc in 1% NEt₃) to yield the Intermediate F compound (0.430 g, 56% yield). ¹H NMR (500 MHz, CDCl₃), δ=7.68-7.66 (d, J=7.88, 1H), 7.57-7.54 (t, J=7.88, 1H), 7.05-7.03 (d, J=6.76, 1H), 6.93-6.90 (t, J=7.32, 1H), 6.61-6.59 (d, J=8.45, 1H), 4.51 (s, 2H), 2.09 (s, 3H), 2.02 (s, 6H). The chemical structure of Intermediate F is shown below:

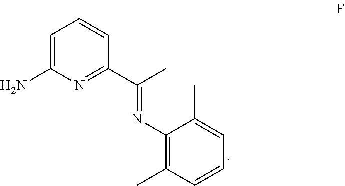 [SMILES: C/C(=N\c1c(C)cccc1C)c1cccc(N)n1]

Intermediate G (3-(1-(o-tolylimino)ethyl) aniline) was prepared as follows. In a round bottom flask, a solution of 1-(6-aminopyridin-2-yl) ethan-1-one (1 g, 7.35 mmol) was added to 2-methylaniline (1.18 g, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (3:1 hexane:EtOAc in 1% NEt₃) to yield the Intermediate G compound (0.148 g, 9.0% yield). ¹H NMR (500 MHZ, CDCl₃) δ=7.65-7.63 (m, 1H), 7.57-7.53 (m, 1H), 7.39 (s, 1H), 7.36-7.29 (m, 2H), 6.75-6.73 (d, J=8.08, 1H), 6.69-6.67 (d, J=8.08, 1H), 6.60-6.58 (d, J=8.08, 1H), 4.47 (s, 2H), 2.24 (s, 3H), 2.14 (s, 3H). The chemical structure of Intermediate G is shown below:

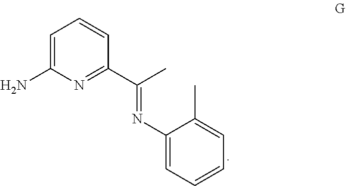 [SMILES: C/C(=N\c1ccccc1C)c1cccc(N)n1]

Intermediate H (6-(1-(p-tolylimino)ethyl) pyridin-2-amine) was prepared as follows. In a round bottom flask, a solution of 1-(6-aminopyridin-2-yl) ethan-1-one (1 g, 7.35 mmol) was added to 4-methylaniline (1.18 g, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo and the residues were purified via flash chromatography (5:1 hexane:EtOAc in 1% NEt₃) to yield the Intermediate H compound (0.3961 g, 23.9% yield). ¹H NMR (500 MHZ, CDCl₃), δ=7.55-7.51 (m, 2H), 7.16-7.14 (d, J=8.65, 2H), 6.72-6.70 (d, J=8.07, 2H), 6.58-6.56 (dt, J=7.00, 1H), 4.48 (s, 2H), 2.35 (s, 3H), 2.26 (s, 3H). The chemical structure of Intermediate H is shown below:

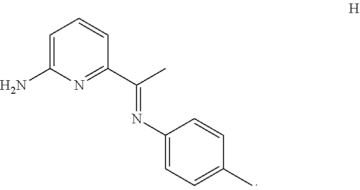 [SMILES: C/C(=N\c1ccc(C)cc1)c1cccc(N)n1]

The Intermediate I, J, K, L, and M compounds were synthesized by the following reaction pathway:

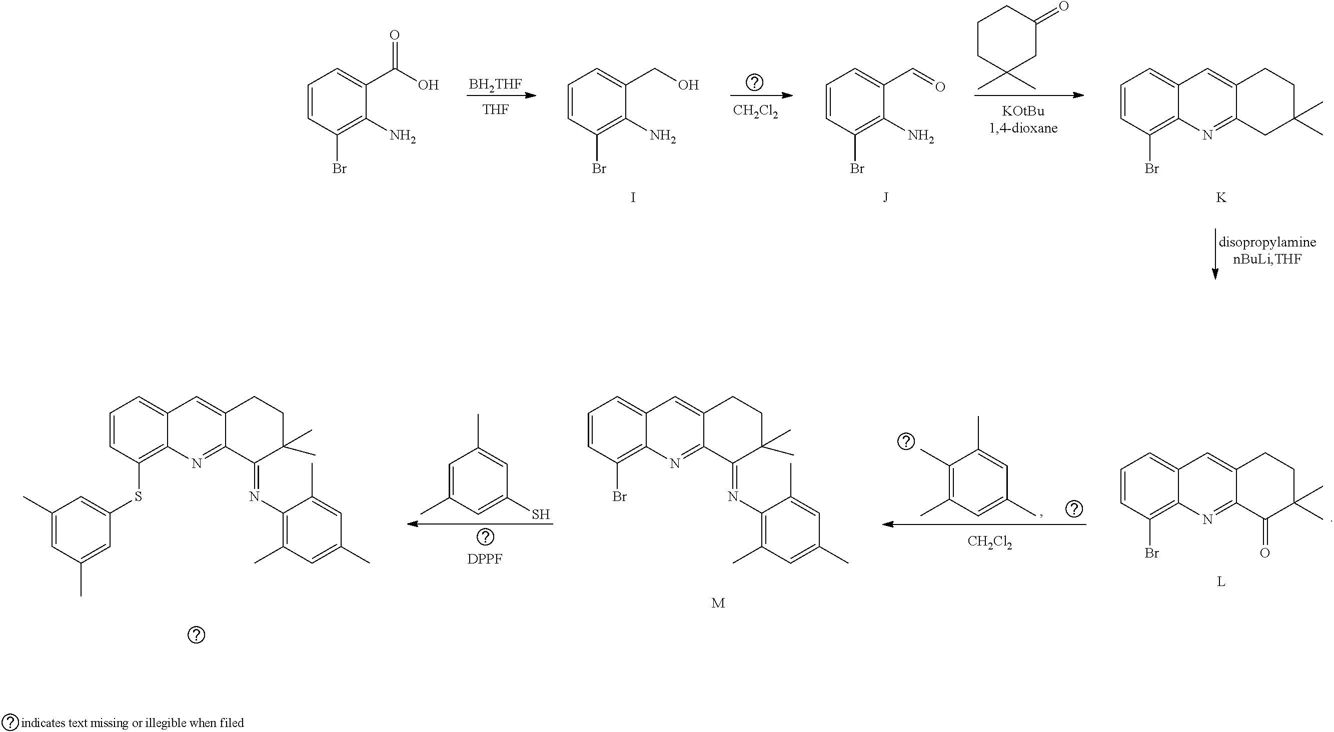

Intermediate I (2-amino-3-bromophenyl) methanol) was prepared as follows. A solution of 2-amino-3-bromobenzoic acid (3 g, 13.9 mmol, 1 equiv.) was prepared in THF (12.5 mL) and stirred at 0° C. BH₃THF (1.0M in THF) (20.8 mL, 1.5 equiv.) was added to the solution dropwise and refluxed for 16 hr. The mixture was filtered and the volatiles were removed in vacuo. The product was purified via flash column chromatography (petroleum ether:ethyl acetate 1:1) to obtain the Intermediate I compound as a white solid (1.9 g, 69% yield). ¹H NMR (500 MHz, CDCl₃), δ=7.41-7.39 (dd, J=1.5, 8.1, 1H), 7.05-7.00 (d, J=7.5, 1H), 6.59-6.55 (t, J=7.8, 1H), 4.73 (brs, 2H), 4.69 (s, 2H).

Intermediate J (2-amino-3-bromobenzaldehyde) was prepared as follows. MnO₂ (1.05 g, 12 mmol, 4 equiv.) was added to a solution of Intermediate I (2-amino-3-bromophenyl) methanol) (610 mg, 3 mmol, 1 equiv.) in CH₂Cl₂ (15.3 mL) and stirred at room temperature for 24 hr. The reaction mixture was filtered, and the volatiles removed in vacuo to obtain the Intermediate J compound (0.572 g, 95% yield). ¹H NMR (500 MHZ, CDCl₃), δ=9.83 (s, 1H), 7.63-7.61 (dd, J=1.4, 7.9, 1H), 7.49-7.47 (dd, J=1.2, 7.7, 1H), 6.69-6.22 (t, J=7.8, 1H), 6.67 (brs, 2H).

Intermediate K (5-bromo-3,3-dimethyl-1,2,3,4-tetrahydroacridine) was prepared as follows. A solution of Intermediate J (2-amino-3-bromobenzaldehyde) (1.35 g, 6.8 mmol, 1 equiv.) was added to 3,3-dimethylcyclohexan-1-one (1 g, 8.1 mmol, 1.2 equiv.), KOtBu (1.8 g, 16 mmol, 2.4 equiv.), and 1,4-dioxane (34 mL), and refluxed for 1 hr. The reaction mixture was concentrated, rinsed with Et₂O, and filtered. Purification by column chromatography (hexanes:EtOAc 10:1) yielded the Intermediate K compound as a yellow solid (1.8 g, 91% yield). ¹H NMR (500 MHZ, CDCl₃), δ=7.95-7.93 (dd, J=1.1, 7.5, 1H), 7.84 (s, 1H), 7.69-7.67 (d, J=8.2, 1H), 7.29-7.26 (t, J=7.9, 1H), 3.06-3.03 (t, J=6.9, 2H), 3.00 (s, 2H), 1.71-1.69 (t, J=6.9, 2H), 1.08 (s, 6H).

Intermediate L (5-bromo-3,3-dimethyl-2,3-dihydroacridin-4 (1H)-one) was prepared as follows. Under an inert atmosphere, diisopropylamine (0.58 mL, 4.3 mmol, 2.5 equiv.) was cooled to −15° C. and n-butyllithium (1.38 mL, 3.4 mmol, 2 equiv.) was added dropwise. The reaction was stirred for 15 min and then THF was added (10 mL). The reaction was warmed to room temperature and stirred for 1 hr. The reaction was then cooled to −15° C. and a solution of Intermediate K (5-bromo-3,3-dimethyl-1,2,3,4-tetrahydroacridine) (0.500 g, 1.7 mmol, 1 equiv.) in THF (15 mL) was slowly added. The reaction was stirred at room temperature for 3 hr. The reaction was then cooled to −15° C. and isoamyl nitrate (2.3 mL, 11.7 mmol, 10 equiv.) was added and stirred at room temperature for 4 hr. The reaction was quenched with H₂O, and extracted with DCM, then concentrated and HCl (18 mL) was added and refluxed overnight. The mixture was neutralized with a NaOH solution. The reaction was extracted with DCM. The volatiles were removed in vacuo and purified by column chromatography (3:1 PE:EtOAc) to yield the Intermediate L compound (0.315 g, 60% yield). ¹H NMR (500 MHZ, CDCl₃), δ=8.11 (s, 1H), 8.07-8.05 (dd, J=1.0, 7.5, 1H), 7.77-7.76 (d, J=8.5, 1H), 7.46-7.42 (t, J=8.0, 1H), 3.25-3.22 (t, J=6.6, 2H), 2.12-2.10 (t, J=6.5, 2H), 1.34 (s, 6H).

Intermediate M (E)-5-bromo-N-mesityl-3,3-dimethyl-2,3-dihydroacridin-4 (1H)-imine) was prepared as follows. A solution of Intermediate L (5-bromo-3,3-dimethyl-2,3-dihydroacridin-4 (1H)-one) (300 mg, 0.98 mmol, 1 equiv.) in CH₂Cl₂ (10 mL) was prepared and distilled TiCl₄ (0.11 mL, 0.98 mmol, 1 equiv.) was added to the solution at 0° C. and stirred for 10 min. At 0° C., 2,4,6-trimethylaniline (0.300 g, 0.98 mmol, 1 equiv.) was added to the solution (0.98 mmol, 1 equiv.) and stirred for 10 min, followed by addition of triethylamine (0.1 mL). The reaction was warmed to room temperature and stirred overnight. The reaction mixture was quenched with NH₄Cl and extracted with DCM and washed with brine. The volatiles were then removed in vacuo. The product was then purified via flash chromatography (petroleum ether:ethyl acetate, 50:1) to yield the Intermediate M compound (0.294 g, 71% yield). ¹H NMR (500 MHZ, CDCl₃), δ 7.93 (s, 1H), 7.81-7.79 (d, J=7.5, 1H), 7.62-7.60 (dd, J=1.0, 8.3, 1H), 7.26-7.24 (t, J=7.7, 1H), 6.71 (s, 2H), 3.14-3.11 (t, J=6.6, 2H), 2.20 (s, 3H), 2.08-2.06 (t, J=6.6, 2H), 1.87 (s, 6H), 1.45 (s, 6H).

Intermediate N (1-(8-Bromo-2-quinolinyl) ethenone) was prepared as follows: Under an inert atmosphere, a 250 mL Schlenk flask was loaded with dry and deoxygenated THF (15 mL) followed by addition of ethylvinylether (4.6 mL, 47 mmol, 4.0 equiv.). The solution was cooled to −40° C. and n-BuLi (2.5 M in hexanes, 9.5 mL, 23.7 mmol, 2 equiv.) was added dropwise. After complete addition, the reaction was warmed to room temperature over 15 min and stirred at room temperature for 1 h. The Schlenk flask was then cooled to −40° C. and dry ZnBr₂ (0.5 M in THF, 47 mL, 23.7 mmol, 2.0 equiv.) was added. The reaction mixture was warmed to room temperature over 15 min and stirred at room temperature for 15 additional min. Pd₂dba₃ (123 mg, 0.107 mmol, 0.9 mol %) and triphenylphosphine (118 mg, 0.450 mmol, 3.8 mol %) were dissolved in dry and deoxygenated THF (15 mL) and added to the reaction mixture at room temperature for 15 min. A solution of 2,8-dibromoquinoline (3.40 g, 11.9 mmol, 1.0 equiv.) in dry and deoxygenated THF (15 mL) was then added at room temperature and stirred for 16 h. The reaction mixture was heated to 65° C. for 4 hr. Then, aqueous HCL (1M, 25 mL) was added, the reaction was stirred for another 4 hr at 65° C. The reaction was cooled to room temperature and quenched by addition of saturated aqueous NaHCO₃ (5 mL), water (15 mL), and tertbutylmethylether (TBME) (2×25 mL). The organic layer was collected, and the aqueous layer extracted with TBME (2×25 mL). The combined organic layers were dried over MgSO₄ and concentrated under reduced pressure. The brown residue was purified via column chromatography (SiO₂, eluent: toluene, the third spot on the TLC plate corresponds to the product) to yield the Intermediate N compound as a tan-yellow solid (1.34 g, 45% yield). ¹H NMR (400 mHz, CDCl₃): 8.24 (d, 1H), 8.14 (d, 1H), 8.09 (d, 1H), 7.81 (d, 1H), 7.47 (t, 1H), 2.94 (s, 3H). The chemical structure of Intermediate N is shown below:

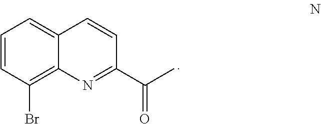

Alternatively, the Intermediate N compound can be synthesized through the use of Intermediate O and Intermediate P compounds as described below.

Intermediate O (8-Bromo-2-quinolinecarboxaldehyde) was prepared as follows. A solution of 8-bromo-2-methylquinoline (0.16 mol) was added to a hot solution of selenium dioxide (0.9 mol) in dioxane and the reaction mixture was heated under reflux for 5 h, then filtered while hot. The solvent was removed in vacuo and purified via column chromatography (dichloromethane) to yield the Intermediate O compound as a pale-yellow solid (93% yield). The chemical structure of Intermediate O is shown below:

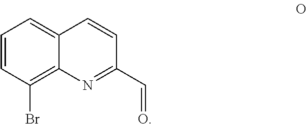 [SMILES: O=Cc1ccc2cccc(Br)c2n1]

Intermediate P (8-Bromo-α-methyl-2-quinolinemethanol) was prepared as follows. A solution of Intermediate O (8-Bromo-2-quinolinecarboxaldehyde) (10.0 mmol, 1.0 equiv.) in THF (100 mL, 0.1 M) was added to an oven dried 250 mL round-bottomed flask under N₂. The mixture was cooled to 0° C. with ice bath, and methyl magnesium bromide (10 mL, 2 M solution in THF, 20.0 mmol, 2.0 equiv.) was added dropwise. The reaction mixture was stirred at 0° C. for 1 h and then warmed to room temperature and stirred for 3 h. The reaction mixture was then cooled to 0° C. and quenched by addition of saturated ammonium chloride solution (40 mL) and brine (100 mL). The aqueous phase was extracted with ethyl acetate (100 mL×2) and the organic phases combined and dried over anhydrous sodium sulfate. The organic fraction was filtered, and the volatiles removed in vacuo. The product was purified via flash column chromatography (silica gel, PE/EtOAc=8:1 to 5:1) to yield the Intermediate P compound as a pale-yellow solid (87%-98% yield). The chemical structure of Intermediate P is shown below:

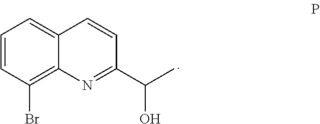

Intermediate N (1-(8-Bromo-2-quinolinyl) ethenone) was alternatively prepared as follows. A solution of Intermediate P (8-Bromo-α-methyl-2-quinolinemethanol) (4.0 mmol, 1.0 equiv.), MnO₂ (2.09 g, 24.0 mmol, 6.0 equiv.) in toluene (40 mL) was added to an oven dried 100 mL round-bottomed flask. The reaction was stirred at room temperature for 20 h. Then, the reaction was filtered and rinsed with ethyl acetate (30 mL×2). The organic phases were combined, and the volatiles removed in vacuo. The product was purified via flash column chromatography (silica gel, PE/EtOAc=8:1) to yield the Intermediate N compound as a pale-yellow solid (90%-95% yield). The chemical structure of Intermediate N is shown below:

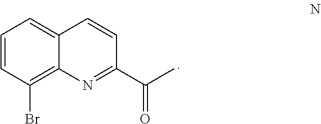

Characterization data for Intermediate compounds N, O, and P can be found, for instance, in Yang, X., Shan, G., Rao, Y., Synthesis of 2-Aminophenols and Heterocycles by Ru-Catalyzed C-H Mono- and Dihydroxylation, Organic Letters 2013, 15 (10), 2334-2337; Sun, H.-R., Zhao, Q., Yang, H., Yang, S., Gou, B.-B., Chen, J., Zhou, L., Chiral Phosphoric-Acid-Catalyzed Cascade Prins Cyclization, Organic Letters 2019, 21 (17), 7143-7148; and Nagy, S., Nifantev, L. E., Neal-Hawkins, K. L., Mihan, S., Catalyst system based on quinoline donors, US Patent Publication 2013/0023634.

The Intermediate Q, R, and S compounds can be synthesized through the use of the Intermediate N compound as described below.

Intermediate Q ((E)-N-(1-8-bromoquinolin-2-yl)ethylidene)-2,4,6-trimethylaniline) was prepared as follows. A solution of 2,4,6-trimethylaniline (1.04 mL, 7.39 mmol), Intermediate N 1-(8-bromoquinlin-2-yl) ethenone (1.54 g, 6.16 mmol) were added to a 25 mL flask and in toluene (10 mL). 4-methylbenzenesulfonic acid (53 mg, 0.308 mmol) was added and the mixture was refluxed under Dean-Stark conditions for 16 h. TLC analysis indicated full conversion of the starting material (SiO₂, eluent: toluene). The reaction solution was cooled to room temperature and filtered over a silica plug eluting with toluene (until no more yellow elutes). The solvent was removed under reduced pressure to yield the Intermediate Q compound as a yellow-orange solid (2.35 g, 99% yield). The chemical structure of Intermediate Q is shown below:

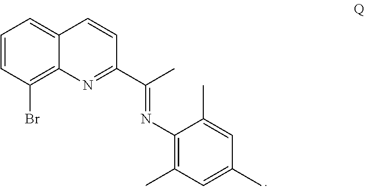 [SMILES: C/C(=N\c1c(C)cc(C)cc1C)c1ccc2cccc(Br)c2n1]

Intermediate R (1-(8-(dicyclohexylphosphino)-2-quinolinyl) ethenone) was prepared as follows. Under inert an atmosphere, a 100 mL flask was charged with diacetoxypalladium (0.056 g, 0.249 mmol, 7.5 mol %), 1,1′-bis(diisopropylphosphino) ferrocene (DiPPF, 0.126 g, 0.227 mmol 9 mol %), sodium tert-butoxide (0.385 g, 4.00 mmol, 1.2 equiv.), and Intermediate N (1.07 g, 4.27 mmol, 1 equiv.), followed by addition of toluene (4 mL, 0.1 M). Dicyclohexylphosphine (0.583 mL, 2.285 mmol, 1.05 equiv.) was added by syringe and stirred for 24 h at 110° C., the reaction was monitored by TCL (SiO₂, toluene). After cooling down to room temperature, degassed sat. aq. NaHCO₃ (10 mL) was added, and extracted. The organic layer was concentrated under reduced pressure affording a black residue. Purification by CC (neutral alumina, toluene/heptane 2:1), 20 g alumina, Fractions 4-11 were collected to yield the Intermediate R compound as a red oil (0.814 g, 55% yield). The chemical structure of Intermediate R is shown below:

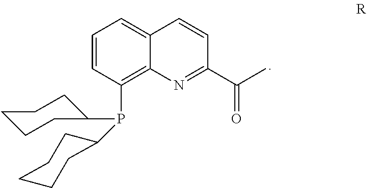 [SMILES: CC(=O)c1ccc2cccc(P(C3CCCCC3)C3CCCCC3)c2n1]

Intermediate S (1-(8-(diphenylphosphino)-2-quinolinyl) ethenone) was prepared as follows. Under inert an atmosphere, a 100 mL flask was charged with diacetoxypalladium (0.056 g, 0.249 mmol, 7.5 mol %), 1,1′-bis(diisopropylphosphino) ferrocene (DiPPF, 0.126 g, 0.277 mmol, 9 mol %), sodium tert-butoxide (0.385 g, 4.00 mmol, 1.2 equiv.), and Intermediate N (1.07 g, 4.27 mmol, 1 equiv.), followed by addition of toluene (4 mL, 0.1 M). Diphenylphosphine (0.583 mL, 3.07 mmol, 1.05 equiv.) was added by syringe and stirred for 24 h at 110° C., the reaction was monitored by TCL: SiO₂, toluene. After cooling down to room temperature, degassed sat. aq. NaHCO₃ (10 mL) was added and extracted. The organic layer was concentrated under reduced pressure affording a black residue. Purification by CC (neutral alumina, toluene/heptane 2:1), 20 g alumina afforded the Intermediate S compound as a brown solid (0.318 g, 45% yield). The chemical structure of Intermediate S is shown below:

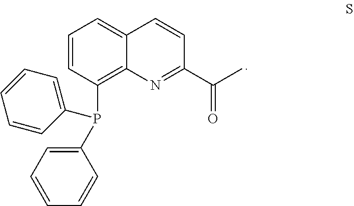 [SMILES: CC(=O)c1ccc2cccc(P(c3ccccc3)c3ccccc3)c2n1]

### Synthesis of Heteroatomic Ligand Compounds

The following heteroatomic ligand compounds were synthesized according to the procedures below.

Ligand 1 ((E)-N-(1-8-(diphenylphosphino)-2-yl)ethylidene)-2,4,6-trimethylaniline) was prepared as follows. Under an inert atmosphere, a 100 mL flask was charged with diacetoxypalladium (0.056 g, 0.251 mmol, 7.5 mol %) 1,1′-1,1′ bis(diisopropylphosphino) ferrocene (DiPPF, 0.126 g, 0.301 mmol), sodium tert-butoxide (0.385 g, 4.02 mmol), and Intermediate Q (1.23 g, 3.35 mmol), followed by addition of toluene (34 mL). The diphenylphosphine (0.583 mL, 3.35 mmol) was added by syringe and stirred for 24 h at 110° C. The reaction mixture was filtered over a celite plug and eluted with toluene (2×20 mL) and the filtrate concentrated under reduced pressure. The residue was further purified by column chromatography (neutral alox, heptane to 3:1 heptane/toluene) to yield the desired Ligand 1 product as a yellow solid (1.13 g, 71% yield). The chemical structure of Ligand 1 is presented below in Table 1.

Ligand 2 ((E)-N-(1-8-(diisobutylphosphino)-2-yl)ethylidene)-2,4,6-trimethylaniline) was prepared as follows. Under an inert atmosphere, a 100 mL flask was charged with diacetoxypalladium (7.5 mol %) 1,1′-bis(diisopropylphosphino) ferrocene (DiPPF, 9 mol %), sodium tert-butoxide (1.2 equiv.), and Intermediate Q (1 g, 3.35 mmol, 1.0 equiv.), followed by addition of toluene (18 mL, 0.15 M). The diisobutyl phosphine (1.1 equiv.) was added by syringe and stirred for 16 h at 110° C. The reaction mixture was filtered over celite and concentrated under reduced pressure. The crude product was purified by column chromatography (neutral alumina 20 g; 2:1 hept/tol), to remove the DiPPF impurity, followed by a second column chromatography (silica, 20 g; 20:1 hept/EtOAc), to remove the quinoline impurity, to yield the desired Ligand 2 product (0.447 g, 38% yield and 97% purity). The chemical structure of Ligand 2 is presented below in Table 1.

Ligand 3 ((E)-N-(1-8-(diisopropylphosphino)-2-yl)ethylidene)-2,4,6-trimethylaniline) was prepared as follows. Under an insert atmosphere, a 100 mL flask was charged with diacetoxypalladium (7.5 mol %), 1,1′-bis(diisopropylphosphino) ferrocene (DiPPF, 9 mol %), sodium tert-butoxide (1.2 equiv.), and Intermediate Q (0.863 g, 1.0 equiv.), followed by addition of toluene (16 mL, 0.15 M). The diisopropyl phosphine (1.1 equiv.) was added by syringe and stirred for 16 h at 110° C. The reaction mixture was filtered over celite and concentrated under reduced pressure. The crude product was purified by column chromatography (silica, 20 g; 20:1 hept/EtOAc). The product and impurity have similar R_{f}-values which made the separation challenging. Under these conditions, a fraction was obtained which did not contain the quinoline derived impurity, however the DiPPF coeluted with the desired product (350 mg). To remove the DiPPF impurity, the obtained material was subjected to column chromatography (neutral alumina 20 g, 2:1 hept/tol). This afforded the desired Ligand 3 product (0.249 g, 26% yield and 91% purity). The chemical structure of Ligand 3 is presented below in Table 1.

Ligand 4 ((E)-N-(1-8-(dicyclohexylphosphino)-2-yl)ethylidene)-2,4,6-trimethylaniline) was prepared as follows. Under an inert atmosphere, a 100 mL flask was charged with diacetoxypalladium (5 mol %), 1,1′-bis(diisopropylphosphino) ferrocene (DiPPF, 6 mol %), sodium tert-butoxide (1.2 equiv.), and Intermediate Q (0.549 g, 1 equiv.), followed by addition of toluene (10 mL, 0.13 M). The dicyclohexyl phosphine (1.05 equiv.) was added by syringe and stirred for 16 h at 110° C. Filtration over celite followed by removal of solvent afforded crude product containing 10% of an unidentified side product (no starting material present in the mixture). The crude product was purified by column chromatography (neutral alumina, 2:1 hept/tol). While the DiPPF impurity could be removed with this chromatographic method the quinoline derived impurity remained. It was found that using thoroughly dried silica gel as the stationary phase and heptanes/EtOAc (20:1) as the mobile phase allowed the remaining 10% impurity to be removed. This afforded the desired Ligand 4 product (0.324 g in 49% yield and 99% purity). The chemical structure of Ligand 4 is presented below in Table 1.

Ligand 5 ((E)-N-(1-8-(diphenylphosphino)-2-yl)ethylidene)-2,4-trimethylaniline) was prepared as follows. Under an inert atmosphere, Intermediate S (0.1000 g, 0.281 mmol, 1.0 equiv.) was added to a solution of 2,4-dimethyl aniline (0.04 mL, 0.338 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 5 product as a red solid (220 mg, 82% yield). A cleaner final product was obtained by dissolving the orange solid in anhydrous methanol and crashing out the product in a freezer overnight and collecting the precipitate by filtration. The chemical structure of Ligand 5 is presented below in Table 1.

Ligand 6 ((E)-N-(1-8-(diphenylphosphino)-2-yl)ethylidene)-4-trimethylaniline) was prepared as follows. Under an inert atmosphere, Intermediate S (0.2860 g, 0.805 mmol, 1.0 equiv.) was added to a solution of 4-methyl aniline (0.11 mL, 0.965 mmol, 1.2 eq) in anhydrous toluene, molecular sieves (4 Å, 200 mg) and silica-alumina catalyst support (50 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 6 product as a yellow solid (0.108 g, 86% yield). The chemical structure of Ligand 6 is presented below in Table 1.

Ligand 6-Fe was prepared as follows: Experiment on the feasibility of templated imine condensation to 6-Fe to avoid purification by column chromatography of the labile PIQ-Ligand. A 15 mL vial loaded with p-toluidine (30.2 mg, 0.281 mmol, 1.05 equiv.), Ligand 6 (100 mg, 0.281 mmol, 1.05 equiv.), iron (II) chloride (34.0 mg, 0.268 mmol, 1.0 equiv.) was inertized. Degassed isopropyl alcohol (3.4 mL) was added and the reaction was stirred for 24 h at 50° C. The dark blue mixture was cooled to room temperature, filtered and washed with iPrOAc (3×1 mL). The wet solid was dried under reduced pressure affording the desired Ligand 6-Fe product (Fe4C-1) as a dark blue solid (128 mg, 84% yield). Elemental Analysis: C, 63.08; H, 4.41; Cl, 12.41; Fe, 9.78; N, 4.90; P, 5.42. The chemical structure of Ligand 6-Fe is presented below in Table 1.

Ligand 7 ((E)-N-(1-8-(dicyclohexylphosphino)-2-yl)ethylidene)-2,4-trimethylaniline) was prepared as follows. Under an inert atmosphere, Intermediate R (0.2000 g, 0.544 mmol, 1.0 equiv.) was added to a solution of 2,4-dimethyl aniline (0.08 mL, 0.653 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 7 product as a red solid (0.327 g, 89% yield). The chemical structure of Ligand 7 is presented below in Table 1.

Ligand 8 ((E)-N-(1-8-(dicyclohexylphosphino)-2-yl)ethylidene)-4-trimethylaniline) was prepared as follows. Under an inert atmosphere, Intermediate R (0.2000 g, 0.544 mmol, 1.0 equiv.) was added to a solution of 4-methyl aniline (0.07 mL, 0.652 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 8 product as a yellow solid (0.208 g, 78% yield). The chemical structure of Ligand 8 is presented below in Table 1.

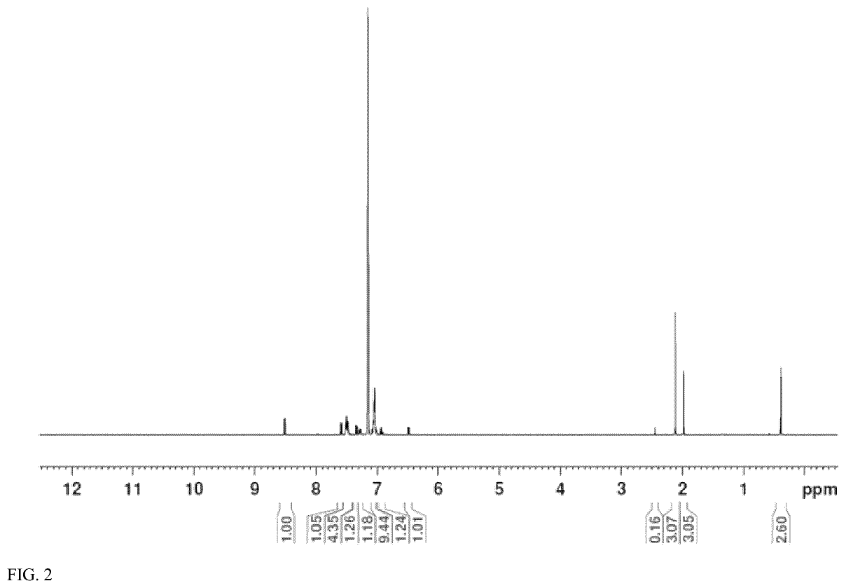

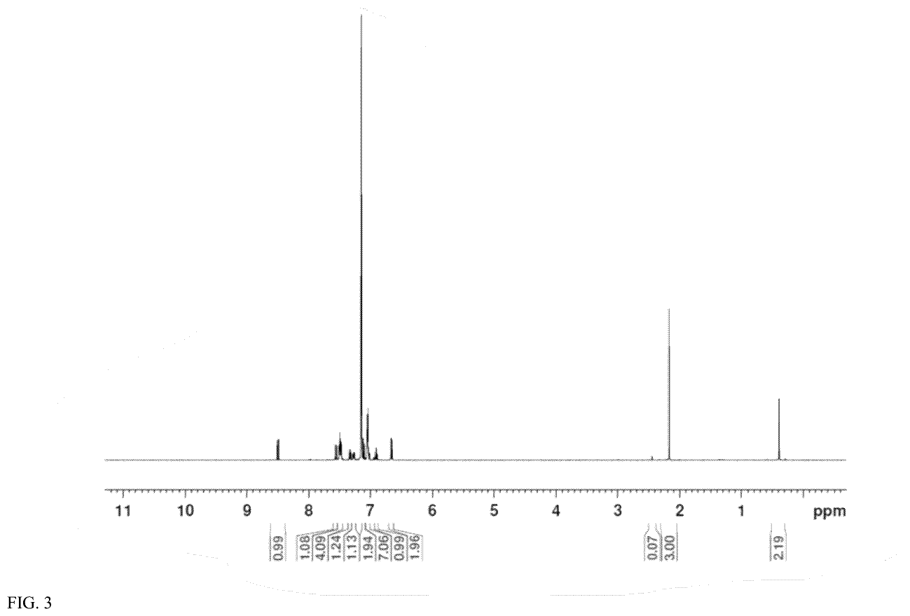

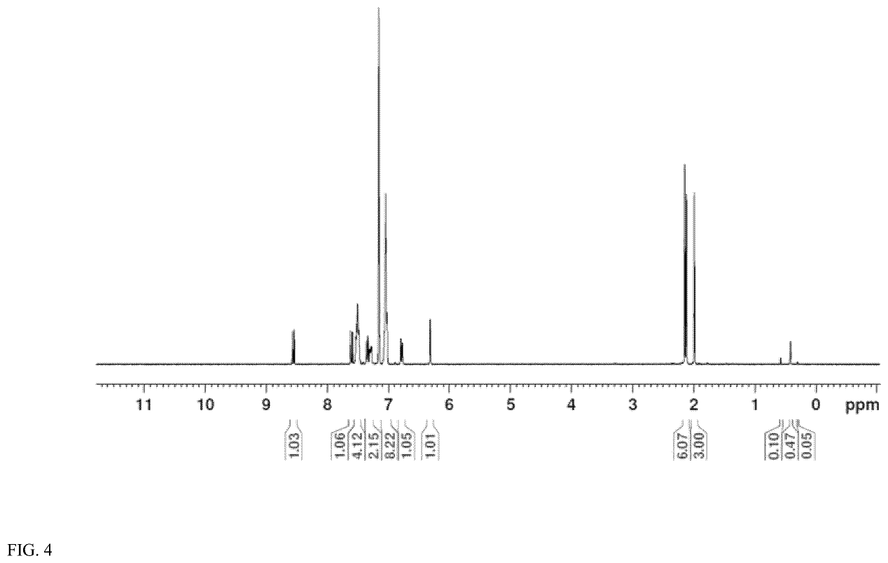

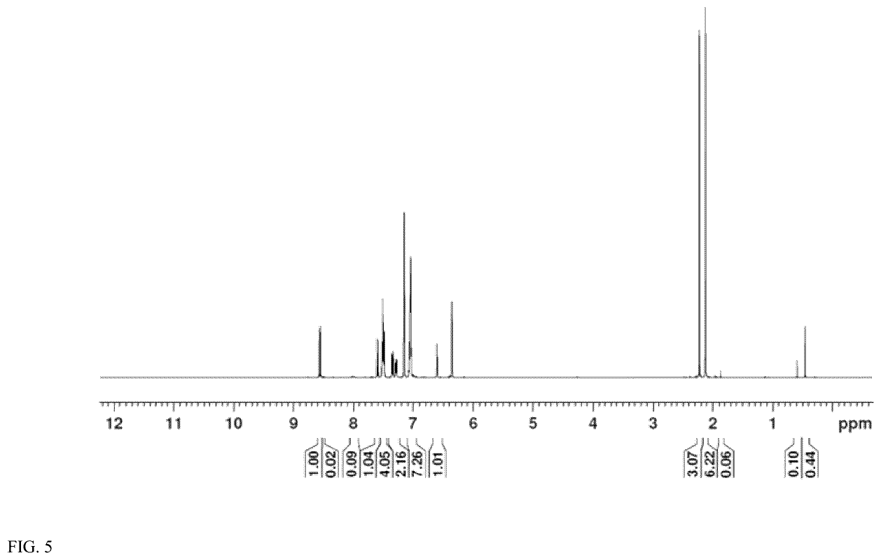

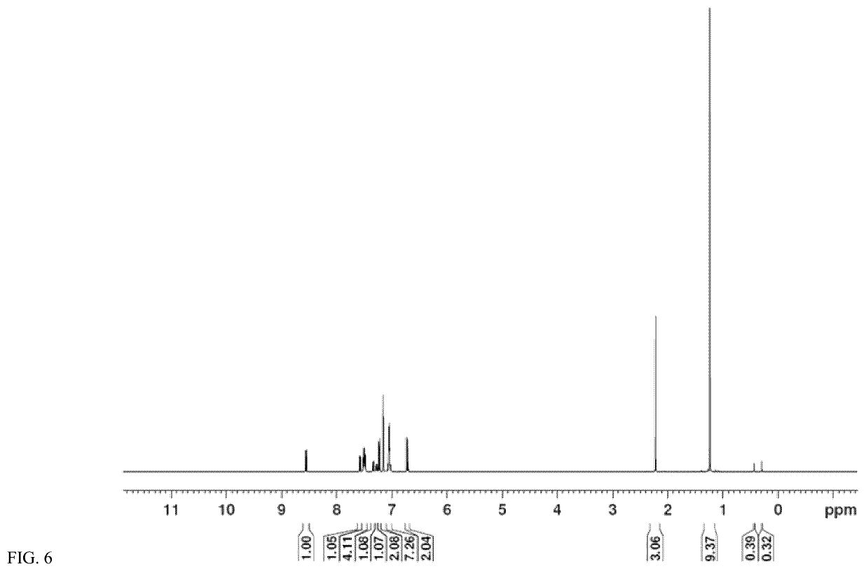

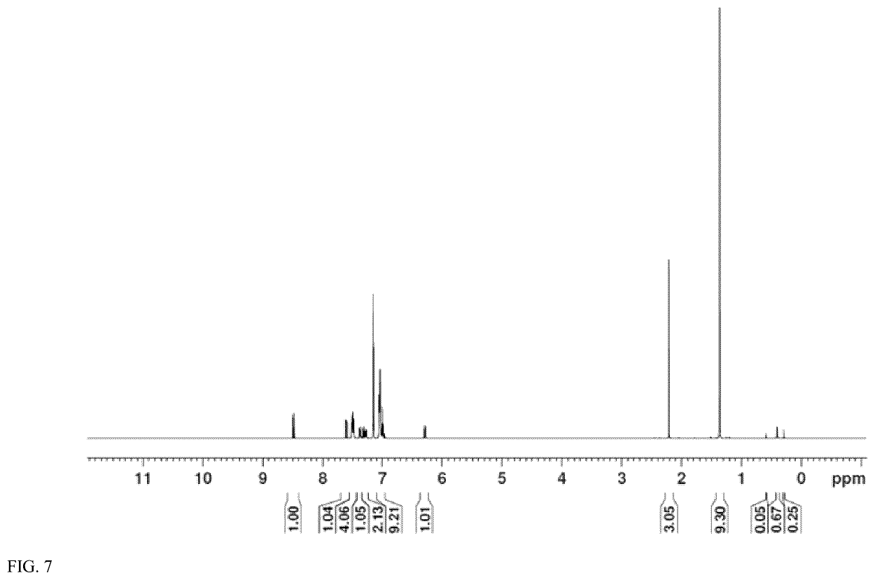

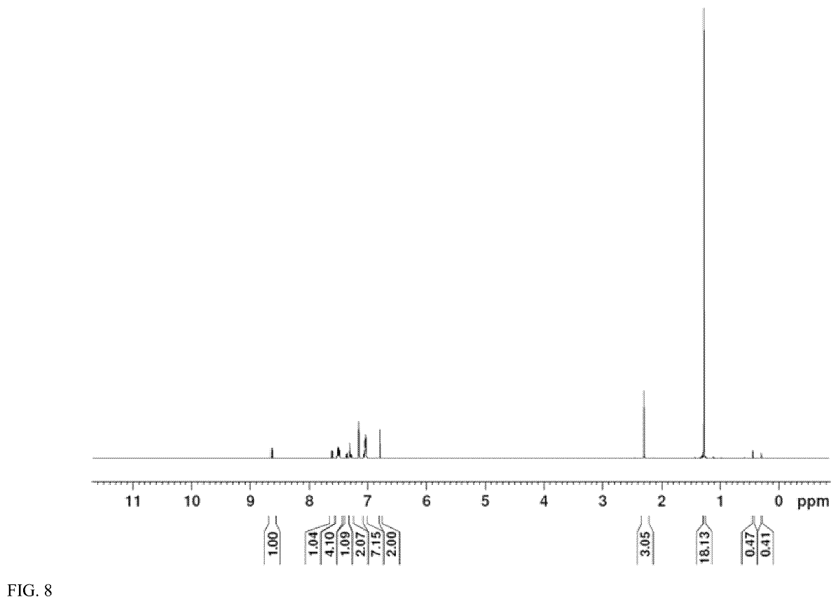

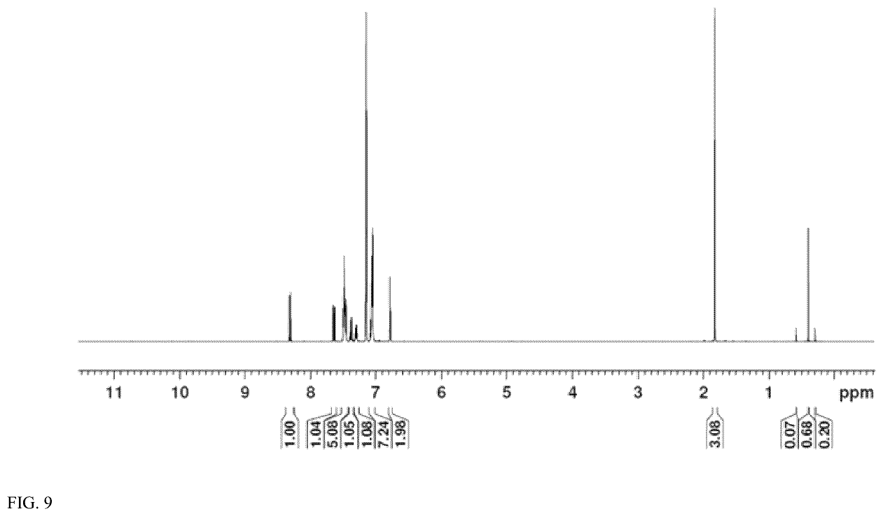

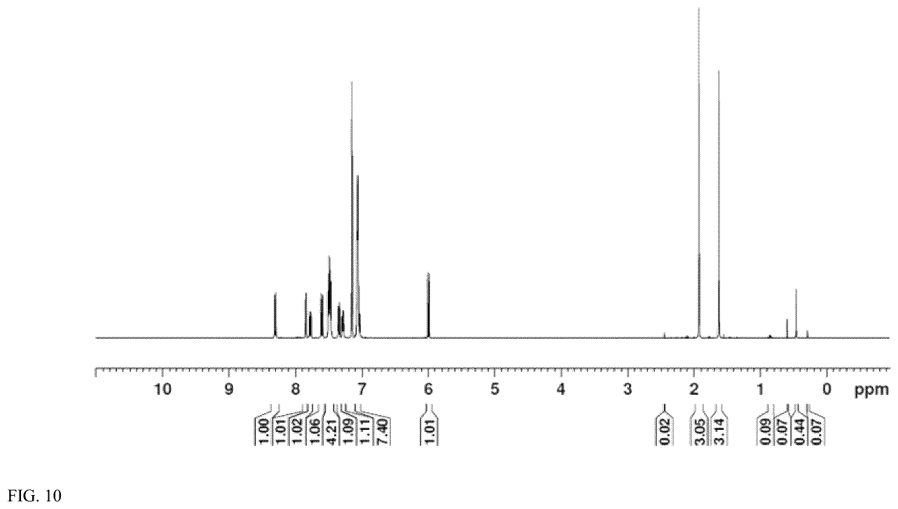

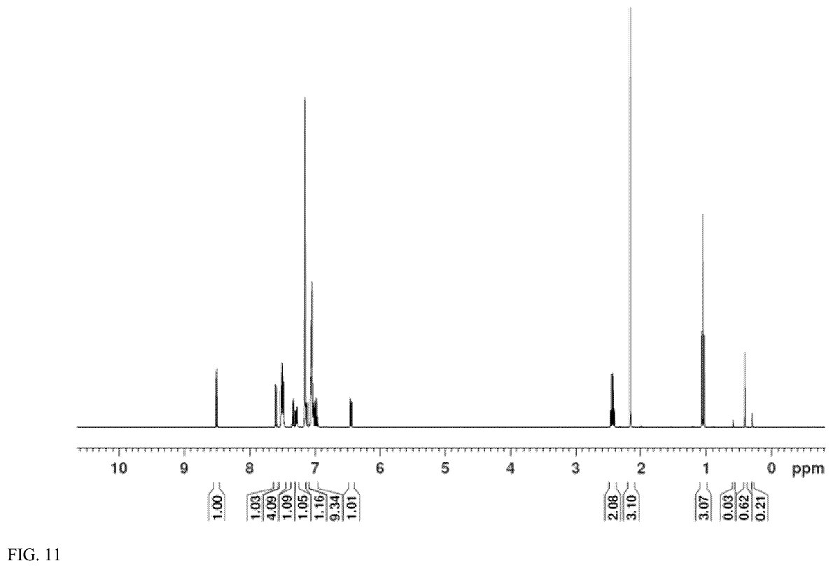

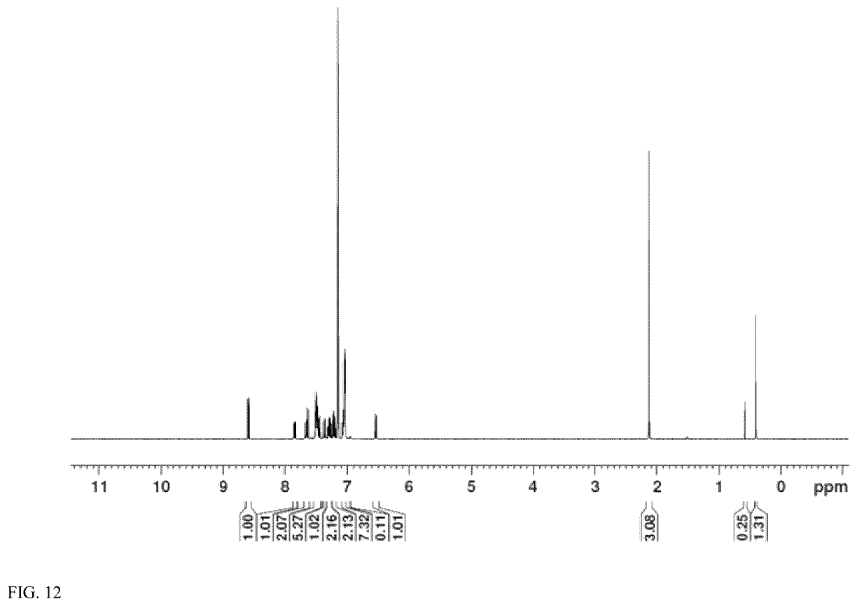

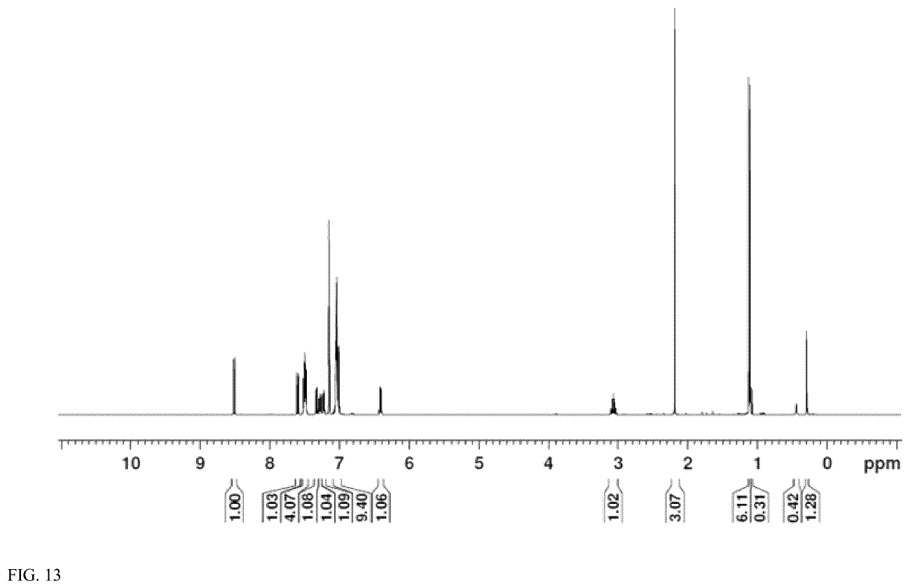

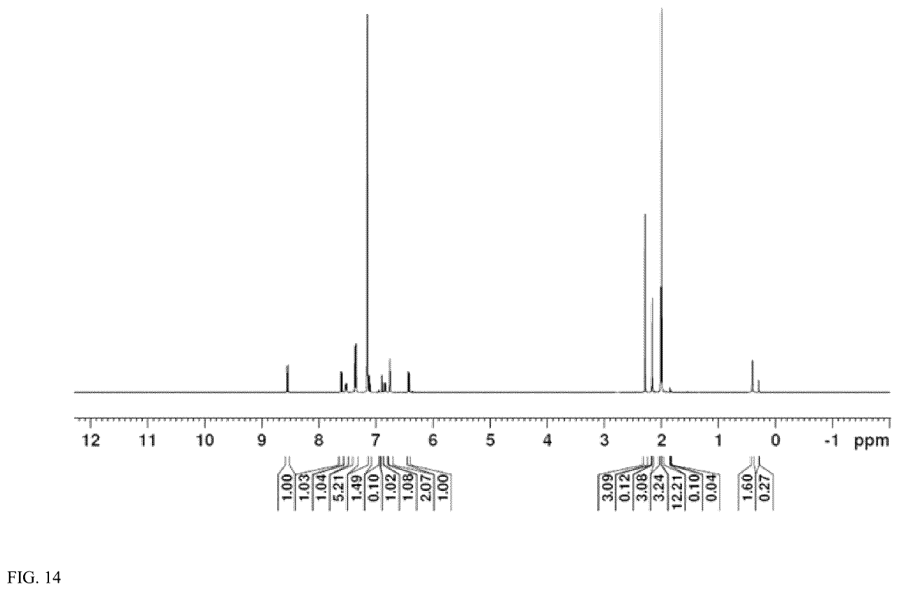

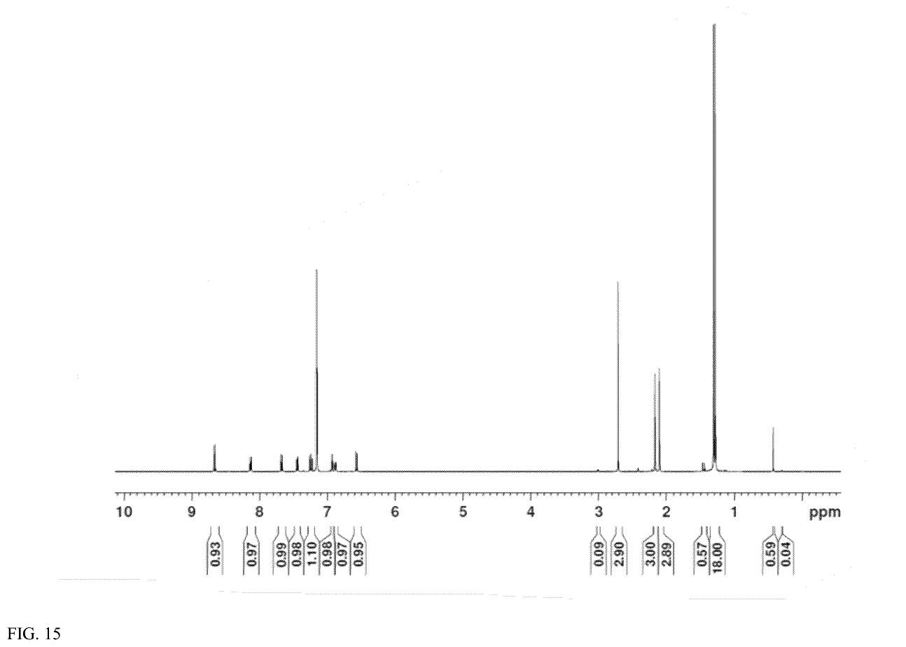

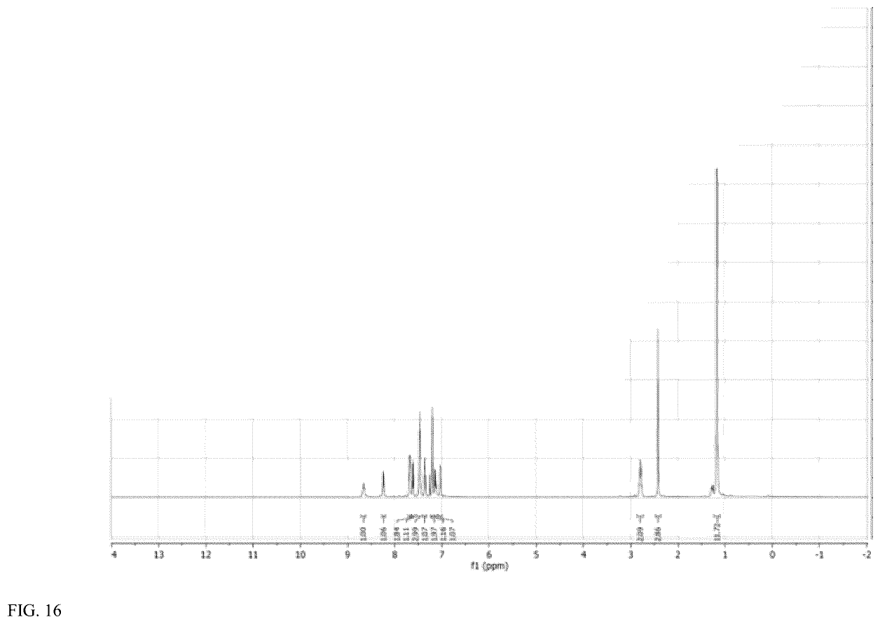

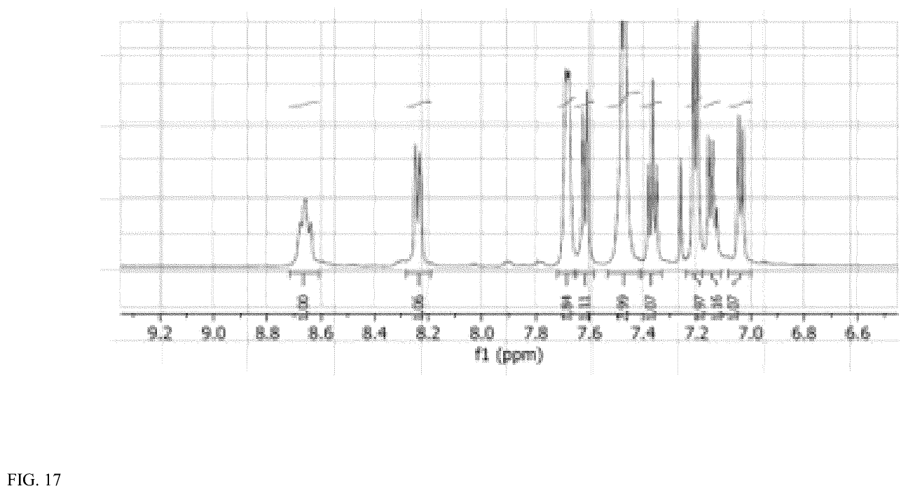

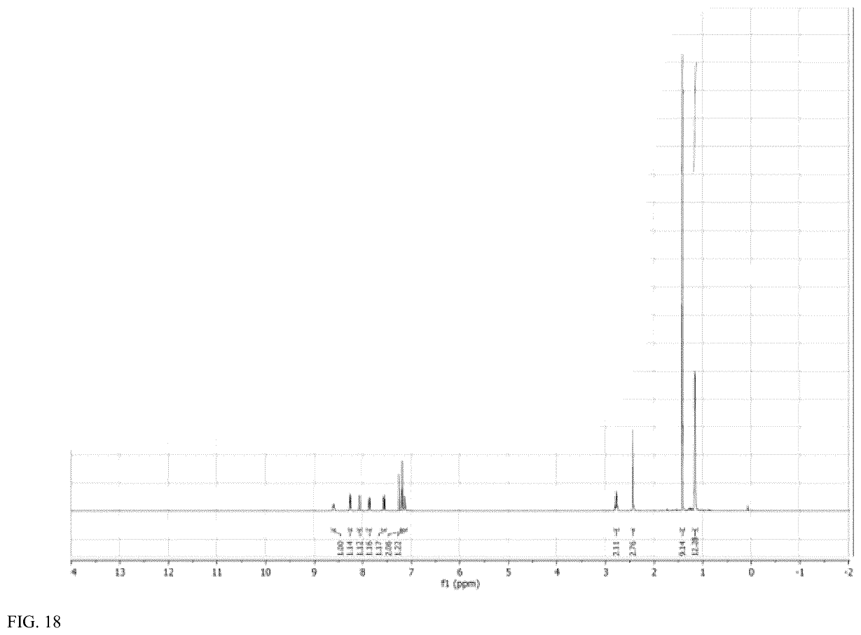

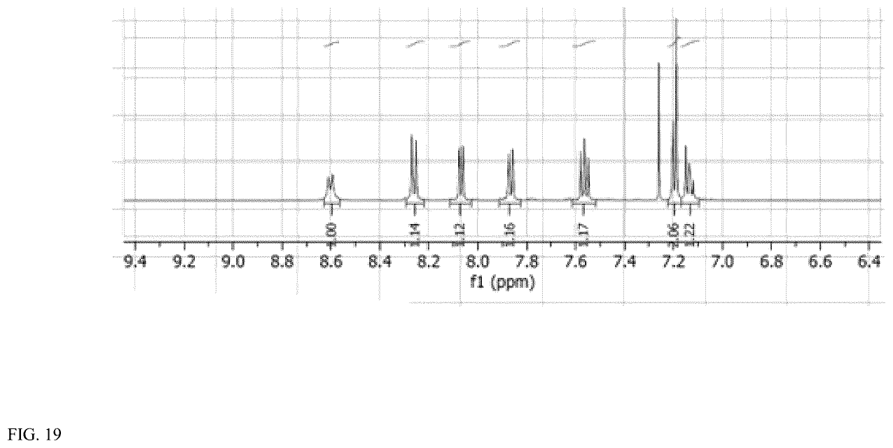

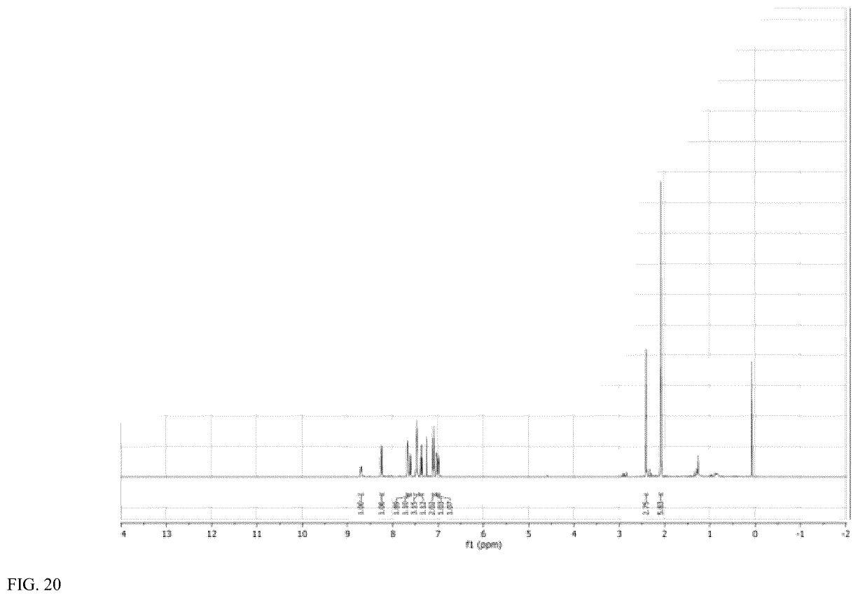

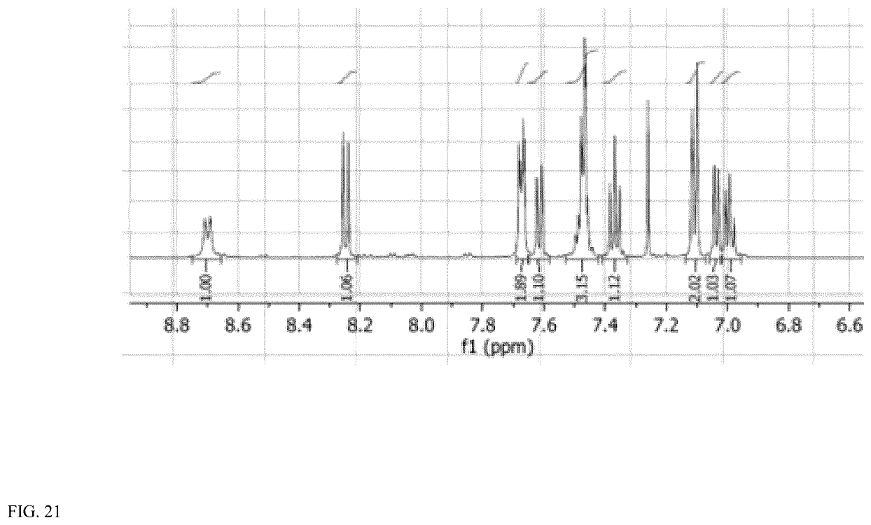

Ligands 9-22 were synthesized using similar techniques to those described hereinabove for Ligands 1-8. The chemical structures for Ligands 9-22 are presented below in Table 1 and ¹H NMR plots confirming the structures for Ligands 9-22 are provided in FIGS. 2-15, respectively. Likewise, Ligands 23-25 were synthesized using similar techniques to those described hereinabove for Ligands 1-8. The chemical structures for Ligands 23-25 are presented below in Table 1 and ¹H NMR plots confirming the structures for Ligands 23-25 are provided in FIGS. 16-17, FIGS. 18-19, and FIGS. 20-21, respectively. Ligand 26 in Table 1 was prepared similarly to Ligands 27-31 described below.

Ligand 27 (1,1-diphenyl-N-(3-(1-(o-tolylimino)ethyl)phenyl)phosphanamine) was prepared as follows. In a glove box, Intermediate G (148 mg, 0.66 mmol) was dissolved in 3 mL of THF (0.2 M) and degassed NEt₃ (0.11 mL, 0.79 mmol, 1.2 equiv.) was added, followed by chlorodiphenylphosphine (0.13 mL, 0.73 mmol, 1.1 equiv.). The reaction mixture was stirred for 16 hr at room temperature. Following filtration, removal of solvent in vacuo yielded the desired Ligand 27 product as a yellow oil (214.0 mg, 80% yield). ¹H NMR (500 MHZ, CDCl₃), δ=7.81-7.78 (m, 2H), 7.73-7.70 (m, 1H), 7.60-7.58 (m, 2H), 7.50-7.49 (m, 5H), 7.38 (m, 6H), 7.03-7.01 (d, J=7.99, 1H), 6.84-6.82 (d, J=8.52, 1H), 6.61-6.57 (m, 2H), 5.28-5.26 (d, J=9.03 1H), 1.85 (s, 3H), 1.55 (s, 3H); ³¹P NMR (202 MHz, CDCl₃), δ 59.9, 27.2. The chemical structure of Ligand 27 is presented below in Table 1.

Ligand 28 (N-(3-(1-((2,4-dimethylphenyl)imino)ethyl)phenyl)-1,1-diphenylphosphanamine was prepared as follows. In a glove box, Intermediate E (148 mg, 0.66 mmol) was dissolved in 3 mL of THF (0.2 M) and degassed NEt₃ (0.11 mL, 0.79 mmol, 1.2 equiv.) was added, followed by chlorodiphenylphosphine (0.13 mL, 0.73 mmol, 1.1 equiv.). The reaction mixture was stirred for 16 hr at room temperature. Following filtration, removal of solvent in vacuo yielded the desired Ligand 28 product as a yellow oil (0.214 g, 80% yield). ¹H NMR (500 MHZ, CDCl₃), δ=7.70-7.68 (d, J=6.91, 1H), 7.60-7.57 (t, J=8.24, 2H), 7.51-7.48 (m, 5H), 7.39-7.37 (m, 6H), 7.09-7.07 (d, J=8.46, 1H), 7.01 (s, 1H), 6.97-6.95 (d, J=7.62, 1H), 6.54-6.52 (d, J=8.04, 1H), 5.26-5.24 (d, J=8.73, 1H), 2.30 (s, 3H), 2.13 (s, 3H), 2.05 (s, 3H); ³¹P NMR (202 MHZ, CDCl₃), δ 59.9, 27.1. The chemical structure of Ligand 28 is presented below in Table 1.

Ligand 29 (N-(3-(1-(mesitylimino)ethyl)phenyl)-1,1-diphenyl phosphanamine) was prepared as follows. In a glove box, Intermediate D (250 mg, 0.987 mmol) was dissolved in 5 mL of THF (0.2 M) and degassed NEt₃ (0.16 mL, 1.18 mmol, 1.2 equiv.) was added, followed by chlorodiphenylphosphine (0.19 mL, 1.09 mmol, 1.1 equiv.). The reaction mixture was stirred for 16 hr at room temperature. Following filtration, removal of solvent in vacuo yielded the desired Ligand 29 product as an orange yellow oil (0.368 g, 85% yield). ¹H NMR (500 MHZ, CDCl₃), δ=7.75-7.73 (d, J=7.6, 1H), 7.61-7.58 (t, J=8.14, 1H), 7.50 (m, 5H), 7.38-7.37 (m, 6H), 7.11-7.09 (d, J=8.14, 1H), 6.85 (s, 2H), 5.28-5.26 (d, J=7.25, 1H), 2.27 (s, 3H), 2.02 (s, 3H), 1.97 (s, 6H); ³¹P NMR (202 MHZ, CDCl₃), δ 60.6, 27.1. The chemical structure of Ligand 29 is presented below in Table 1.

Ligand 30 (N-(3-(1-((2,6-dimethylphenyl)imino) ethyl)phenyl)-1,1 diphenylphosphanamine) was prepared as follows. In a glove box, Intermediate F (250 mg, 1.05 mmol) was dissolved in 5.3 mL of THF and degassed NEt₃ (0.17 mL, 1.25 mmol, 1.2 equiv.) was added followed by chlorodiphenylphosphine (0.21 mL, 1.15 mmol, 1.1 equiv.). The reaction mixture was stirred for 16 hr at room temperature. Following filtration, removal of solvent in vacuo yielded the desired Ligand 30 product as a yellow oil (0.400 g, 44.4% yield). ¹H NMR (500 MHZ, CDCl₃), δ 7.78-7.76 (d, J=5.5, 1H), 7.62-7.59 (t, J=7.9, 1H), 7.50-7.48 (m, 6H), 7.39-7.36 (m, 4H), 7.04-7.02 (d, J=7.5, 2H), 7.00-6.98 (d, J=7.5, 1H), 6.92-6.89 (t, J=7.4, 1H), 5.28-5.26 (d, J=7.5, 1H), 2.03 (s, 3H), 2.01 (s, 3H), 1.93 (s, 1H); ³¹P NMR (202 MHZ, CDCl₃), δ 60.63, 27.14. The chemical structure of Ligand 30 is presented below in Table 1.

Ligand 31 (1,1-diphenyl-N-(3-(1-(p-tolylimino)ethyl) phenyl)phosphanamine) was prepared as follows. In a glove box, Intermediate H (250 mg, 1.11 mmol) was dissolved in 5.6 mL of THF and degassed NEt₃ (0.19 mL, 1.33 mmol, 1.2 equiv.) was added, followed by chlorodiphenylphosphine (0.22 mL, 1.22 mmol, 1.1 equiv.). The reaction mixture was stirred for 16 hr at room temperature. Following filtration, removal of solvent in vacuo yielded the desired Ligand 31 product as a yellow oil (0.349 g, 64% yield). ¹H NMR (500 MHz, CDCl₃), δ=7.63-7.61 (d, J=8.55, 1H), 7.59-7.56 (t, J=7.48, 1H), 7.49-7.48 (m, 5H), 7.38-7.37 (m, 6H), 7.15-7.13 (d, J=8.0, 2H), 6.70-6.68 (d, J=8.2, 2H), 6.63-6.61 (d, J=8.2, 1H), 5.29 (brs, 1H), 2.34 (s, 3H), 2.19 (s, 3H); ³¹P NMR (202 MHZ, CDCl₃), δ 59.82, 27.24. The chemical structure of Ligand 31 is presented below in Table 1.

Ligand 34 ((E)-5-((3,5-dimethylphenyl)thio)-N-mesityl-3,3-dimethyl-2,3-dihydroacridin-4 (1H)-imine) was prepared as follows. In a glove box, Intermediate M ((E)-5-bromo-N-mesityl-3,3-dimethyl-2,3-dihydroacridin-4 (1H)-imine) (100 mg, 0.25 mmol, 1 equiv.), Pd(OAc)₂ (2.9 mg, 5 mol %), DiPPF (8.5 mg, 6 mol %), NaOtBu (29.4 mg, 0.3 mmol, 1.2 equiv.) and toluene (2.5 mL) were combined and stirred for 1 h at room temperature. Then, 3,5-dimethylthiophenyl (35.2 mg, 0.25 mmol, 1 equiv.) was added to the reaction solution and stirred for 24 h at 120° C. The solvent was removed in vacuo and purified via flash chromatography (hexanes:EtOAc 5:1) to yield the desired Ligand 34 product as a deep red solid (0.096 g, 81% yield). ¹H NMR (500 MHZ, CDCl₃), δ=7.88 (s, 1H), 7.32-7.30 (d, J=8.1, 1H). 7.16-7.13 (t. J=7.6, 1H). 7.12 (s. 2H), 7.05 (s, 1H). 6.75 (s. 2H), 6.70-6.69 (dd, J=7.5.1.2, 1H). 3.12-3.09 (t. J=6.7, 2H). 2.36 (s, 6H), 2.23 (s, 3H), 2.08-2.05 (t. J=6.7, 2H). 1.90 (s. 6H), 1.46 (s. 6H). The chemical structure of Ligand 34 is shown below:

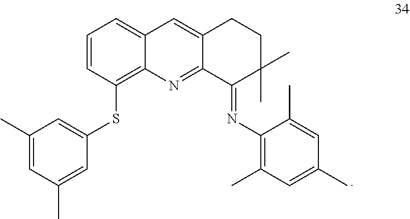

TABLE 1

| | |
|---|---|
| 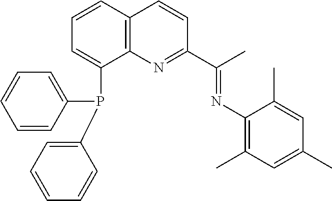 [SMILES: C/C(=N\c1c(C)cc(C)cc1C)c1ccc2cccc(P(c3ccccc3)c3ccccc3)c2n1] | 1 |
| 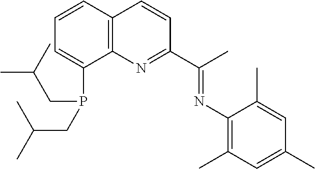 [SMILES: C/C(=N\c1c(C)cc(C)cc1C)c1ccc2cccc(P(CC(C)C)CC(C)C)c2n1] | 2 |
| 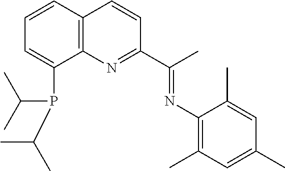 [SMILES: C/C(=N\c1c(C)cc(C)cc1C)c1ccc2cccc(P(C(C)C)C(C)C)c2n1] | 3 |
| 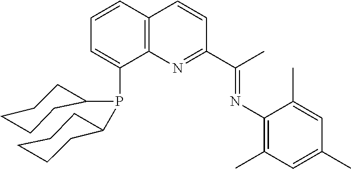 [SMILES: C/C(=N\c1c(C)cc(C)cc1C)c1ccc2cccc(P(C3CCCCC3)C3CCCCC3)c2n1] | 4 |
| 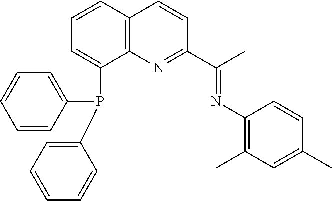 [SMILES: C/C(=N\c1ccc(C)cc1C)c1ccc2cccc(P(c3ccccc3)c3ccccc3)c2n1] | 5 |
| 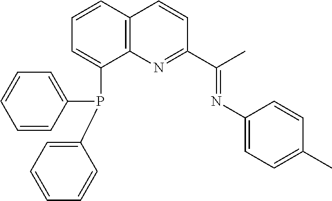 [SMILES: C/C(=N\c1ccc(C)cc1)c1ccc2cccc(P(c3ccccc3)c3ccccc3)c2n1] | 6 |
| 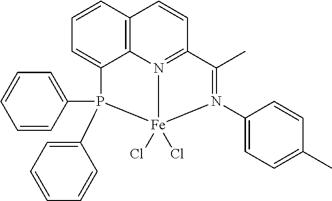 | 6•Fe |
| 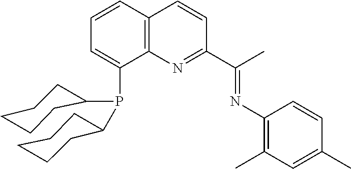 [SMILES: C/C(=N\c1ccc(C)cc1C)c1ccc2cccc(P(C3CCCCC3)C3CCCCC3)c2n1] | 7 |
| 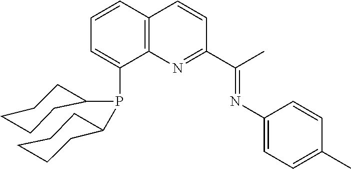 [SMILES: C/C(=N\c1ccc(C)cc1)c1ccc2cccc(P(C3CCCCC3)C3CCCCC3)c2n1] | 8 |
| 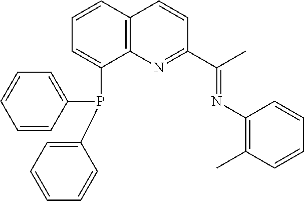 [SMILES: C/C(=N\c1ccccc1C)c1ccc2cccc(P(c3ccccc3)c3ccccc3)c2n1] | 9 |
| 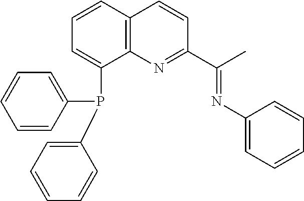 [SMILES: C/C(=N\c1ccccc1)c1ccc2cccc(P(c3ccccc3)c3ccccc3)c2n1] | 10 |
| 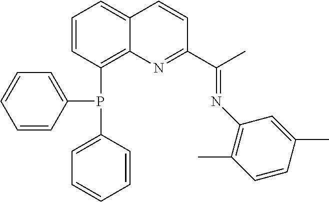 [SMILES: C/C(=N\c1cc(C)ccc1C)c1ccc2cccc(P(c3ccccc3)c3ccccc3)c2n1] | 11 |
| 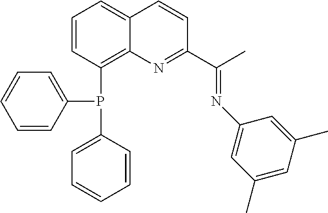 [SMILES: C/C(=N\c1cc(C)cc(C)c1)c1ccc2cccc(P(c3ccccc3)c3ccccc3)c2n1] | 12 |
| 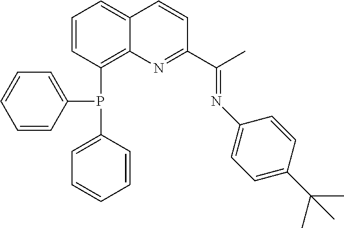 [SMILES: C/C(=N\c1ccc(C(C)(C)C)cc1)c1ccc2cccc(P(c3ccccc3)c3ccccc3)c2n1] | 13 |
| 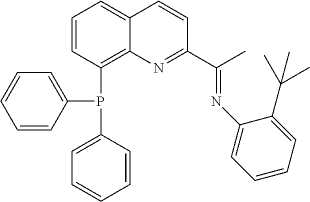 [SMILES: C/C(=N\c1ccccc1C(C)(C)C)c1ccc2cccc(P(c3ccccc3)c3ccccc3)c2n1] | 14 |
| 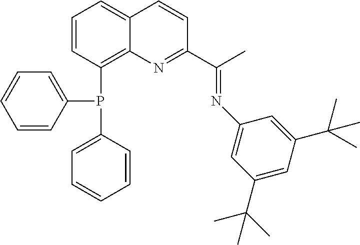 [SMILES: C/C(=N\c1cc(C(C)(C)C)cc(C(C)(C)C)c1)c1ccc2cccc(P(c3ccccc3)c3ccccc3)c2n1] | 15 |
| 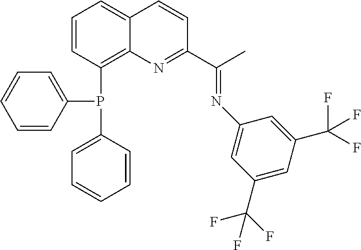 [SMILES: C/C(=N\c1cc(C(F)(F)F)cc(C(F)(F)F)c1)c1ccc2cccc(P(c3ccccc3)c3ccccc3)c2n1] | 16 |
| 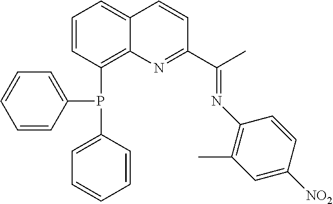 [SMILES: C/C(=N\c1ccc([N+](=O)[O-])cc1C)c1ccc2cccc(P(c3ccccc3)c3ccccc3)c2n1] | 17 |
| 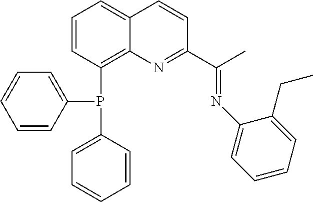 [SMILES: CCc1ccccc1/N=C(\C)c1ccc2cccc(P(c3ccccc3)c3ccccc3)c2n1] | 18 |
| 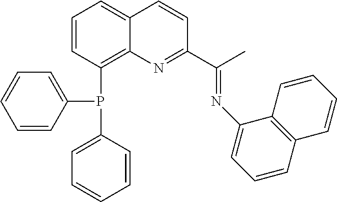 [SMILES: C/C(=N\c1cccc2ccccc12)c1ccc2cccc(P(c3ccccc3)c3ccccc3)c2n1] | 19 |
| 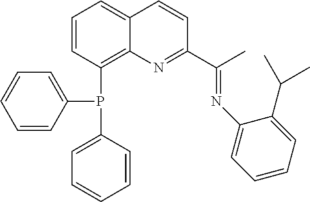 [SMILES: C/C(=N\c1ccccc1C(C)C)c1ccc2cccc(P(c3ccccc3)c3ccccc3)c2n1] | 20 |
| 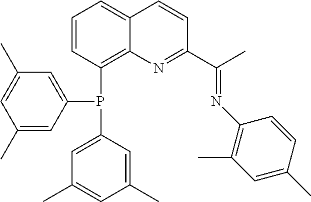 [SMILES: C/C(=N\c1ccc(C)cc1C)c1ccc2cccc(P(c3cc(C)cc(C)c3)c3cc(C)cc(C)c3)c2n1] | 21 |
| 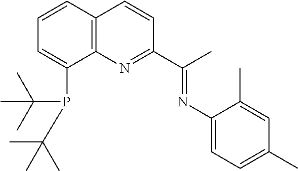 [SMILES: C/C(=N\c1ccc(C)cc1C)c1ccc2cccc(P(C(C)(C)C)C(C)(C)C)c2n1] | 22 |
| 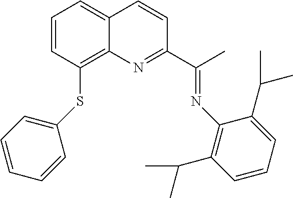 [SMILES: C/C(=N\c1c(C(C)C)cccc1C(C)C)c1ccc2cccc(Sc3ccccc3)c2n1] | 23 |
| 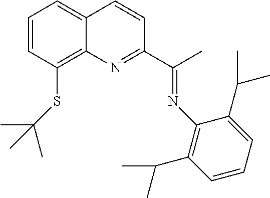 [SMILES: C/C(=N\c1c(C(C)C)cccc1C(C)C)c1ccc2cccc(SC(C)(C)C)c2n1] | 24 |
| 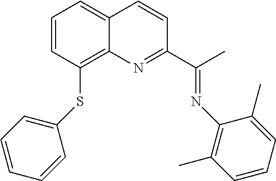 [SMILES: C/C(=N\c1c(C)cccc1C)c1ccc2cccc(Sc3ccccc3)c2n1] | 25 |
| 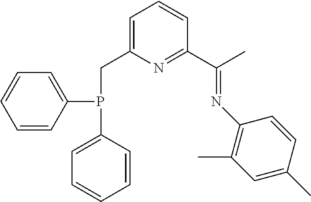 [SMILES: C/C(=N\c1ccc(C)cc1C)c1cccc(CP(c2ccccc2)c2ccccc2)n1] | 26 |
| 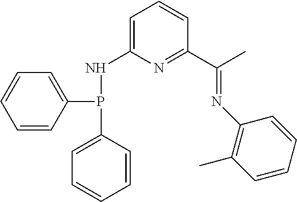 [SMILES: C/C(=N\c1ccccc1C)c1cccc(NP(c2ccccc2)c2ccccc2)n1] | 27 |
| 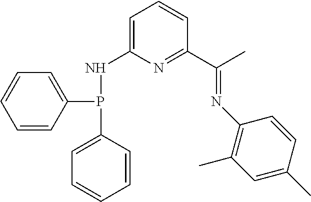 [SMILES: C/C(=N\c1ccc(C)cc1C)c1cccc(NP(c2ccccc2)c2ccccc2)n1] | 28 |
| 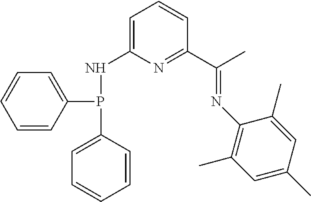 [SMILES: C/C(=N\c1c(C)cc(C)cc1C)c1cccc(NP(c2ccccc2)c2ccccc2)n1] | 29 |
| 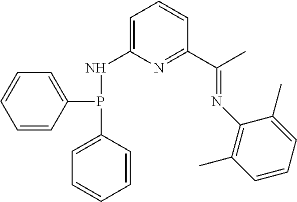 [SMILES: C/C(=N\c1c(C)cccc1C)c1cccc(NP(c2ccccc2)c2ccccc2)n1] | 30 |
| 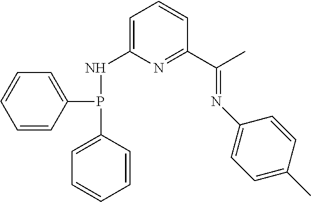 [SMILES: C/C(=N\c1ccc(C)cc1)c1cccc(NP(c2ccccc2)c2ccccc2)n1] | 31 |

### Examples 1-21

The oligomerization experiments of Examples 1-21 were performed as follows. The heteroatomic ligands prepared above, and shown in Table 1, and an excess of a representative soluble iron source (transition metal compound) were combined in a small amount of an aromatic or aliphatic hydrocarbon solvent such as toluene, xylene, or cyclohexane, and added to a sealed NMR tube, which was then affixed to the impeller shaft of a high pressure autoclave, according to the procedure described in Organometallics 2003, 22, 3178 (Small). Cyclohexane solvent (200 mL) and MMAO-3A (modified MAO) were added to the sealed, evacuated autoclave, the reactor was pressurized with ethylene (400-800 psig range), and stirring was begun to break the glass and begin the reaction. Reaction initial (Tᵢₙᵢₜᵢₐₗ) and maximum (Tₘₐₓ) temperatures are indicated in Table 2. Ethylene was fed on demand, and the reactions were terminated by degassing after 15 min. Products were analyzed by gas chromatography, using an internal standard.

The ethylene oligomerization experiments of Examples 1-21 are summarized in Table 2. Yields of volatile products (i.e. C₄) were extrapolated using the Schulz-Flory constant K, with total yields and productivities based on the C₄-C₂₆ products. In some cases, the Schulz-Flory constant is known to drift, typically upwards with increasing carbon number. Therefore, the extrapolation for calculating the K value for C₆/C₄ was based on the rate of change for the three prior fraction measurements. For example, K values for C₁₂/C₁₀, C₁₀/C₈, and C₈/C₆ of 0.52, 0.50, and 0.48, respectively, would give an extrapolated K value of 0.46 for calculating the amount of 1-butene formed.

As shown in Table 2, several examples unexpectedly had molar values of K (nC₁₂/nC₁₀) and K (nC₁₀/nC₈) of 0.5 and below. Further, yields of over 20 g of C₄-C₂₆ products also were achieved, as well as catalyst productivities in the range of 10,000-60,000 g/mmol ligand. The K values for Example 16 (utilizing Ligand 14) were very high (in excess of 0.8-0.9), which typically indicates excessive heavy oligomer or polymer formation. In contrast, the K values for Example 14 (utilizing Ligand 12) were very low (less than 0.2), which typically indicates a preference for dimerization to produce 1-butene. For 1-hexene and 1-octene formation, ordinarily K values in the 0.4-0.6 range are most suitable. Beneficially, several of Examples 1-21 and several different ligands had K values in this general range, such as Examples 6, 9, 11-13, and 17-21.

TABLE 2

| | | Ligand | | | | | K | K | Productivity | Yield |
|---|---|---|---|---|---|---|---|---|---|---|
| | Ligand | MW | MMAO-3A | Metal source | Tᵢₙᵢₜᵢₐₗ | Tₘₐₓ | (nC₁₂/ | (nC₁₀/ | (g/mmol | (g C₄- |
| Example | (mg) | (g/mol) | (Al:Ligand) | (metal:ligand) | (° C.) | (° C.) | nC₁₀) | nC₈) | ligand) | C₂₆) |
| 1 | 1 (0.5) | 470 | 500:1 | Fe(octanoate)₃ | 30 | 62 | 0.786 | 0.767 | 16,290 | 17.3 |
| | | | | (1:1) | | | | | | |
| 2 | 1 (0.5) | 470 | 500:1 | Fe(acac)₂ | 30 | 69 | 0.801 | 0.780 | 12,940 | 13.8 |
| | | | | (4:1) | | | | | | |
| 3 | 2 (0.5) | 432 | 2000:1 | Fe(octanoate)₃ | 30 | 51 | 0.807 | 0.784 | 8,890 | 10.3 |
| | | | | (4:1) | | | | | | |
| 4 | 3 (0.5) | 404 | 2000:1 | Fe(octanoate)₃ | 30 | 44 | 0.851 | 0.825 | 3,690 | 4.6 |
| | | | | (4:1) | | | | | | |
| 5 | 4 (0.48) | 482 | | | 30 | 60 | ND- | | | |
| | | | | | | | waxes | | | |
| 6 | 5 (0.92) | 458 | 1000:1 | Fe(octanoate)₃ | 30 | 90 | 0.514 | 0.489 | 19,100 | 38.2 |
| | | | | (4:1) | | | | | | |
| 7 | 6 (0.89) | 444 | 1000:1 | Fe(octanoate)₃ | 30 | 90 | 0.337 | 0.309 | 26,920 | 53.0 |
| | | | | (4:1) | | | | | | |
| 8 | 5 (0.45) | 458 | 1000:1 | Fe(octanoate)₃ | 75 | 112 | 0.460 | | 30,930 | 30.4 |
| | | | | (4:1) | | | | | | |
| 9 | 7 (0.94) | 470 | 1000:1 | Fe(octanoate)₃ | 30 | 55 | 0.449 | 0.421 | 6,620 | 13.2 |
| | | | | (4:1) | | | | | | |
| 10 | 8 (0.92) | 456 | 1000:1 | Fe(octanoate)₃ | 30 | 82 | 0.194 | 0.157 | 24,350 | 49.1 |
| | | | | (4:1) | | | | | | |
| 11 | 9 (0.43) | 444 | 1000:1 | Fe(octanoate)₃ | 30 | 101 | 0.493 | 0.465 | 26,780 | 26.8 |
| | | | | (4:1) | | | | | | |
| 12 | 9 (0.43) | 444 | 1000:1 | Fe(octanoate)₃ | 75 | 100 | | 0.446 | 30,560 | 30.5 |
| | | | | (4:1) | | | | | | |
| 13 | 11 (0.46) | 458 | 1000:1 | Fe(octanoate)₃ | 75 | 95 | 0.454 | 0.438 | 10,140 | 9.2 |
| | | | | (4:1) | | | | | | |
| 14 | 12 (0.46) | 458 | 1000:1 | Fe(octanoate)₃ | 75 | 125 | 0.199 | 0.171 | 55,240 | 55.3 |
| | | | | (4:1) | | | | | | |
| 15 | 13 (0.49) | 486 | 1000:1 | Fe(octanoate)₃ | 75 | 79 | 0.296 | 0.257 | 2,880 | 2.7 |
| | | | | (4:1) | | | | | | |
| 16 | 14 (0.49) | 486 | 1000:1 | Fe(octanoate)₃ | 75 | 102 | 0.912 | 0.880 | 1,120 | 0.4 |
| | | | | (4:1) | | | | | | |
| 17 | 17 (0.49) | 490 | 1000:1 | Fe(octanoate)₃ | 75 | 103 | 0.605 | 0.559 | 10,850 | 8.1 |
| | | | | (4:1) | | | | | | |
| 18 | 18 (0.46) | 459 | 1000:1 | Fe(octanoate)₃ | 75 | 78 | 0.590 | 0.546 | 3,550 | 2.8 |
| | | | | (4:1) | | | | | | |
| 19 | 27 (0.41) | 409 | 1000:1 | Fe(octanoate)₃ | 75 | 80 | | 0.493 | 6,680 | 6.7 |
| | | | | (4:1) | | | | | | |
| 20 | 28 (0.42) | 423 | 1000:1 | Fe(octanoate)₃ | 75 | 81 | | 0.533 | 13,190 | 13.1 |
| | | | | (4:1) | | | | | | |
| 21 | 29 (0.44) | 438 | 1000:1 | Fe(octanoate)₃ | 75 | 77 | | 0.465 | 11,330 | 11.3 |
| | | | | (4:1) | | | | | | |

### Examples 22-37

The general procedure for producing alkylaluminoxane compositions was as follows. In a drybox, a stirbar was added to a three-necked 1-liter flask. The flask was fitted with a thermocouple on one opening, a vent line on another opening, and a third opening was used to add the reagents. At room temperature (˜23° C.), trimethylaluminum (TMA) and triethylaluminum (TEA), either neat or in aliphatic or aromatic diluents, respectively, were added to the flask, followed by additional diluent to target a final weight percent aluminum in the finished product. Stirring was begun, making sure that the tip of the thermocouple was submerged in the liquid, and a septum was placed on the third neck of the flask. The appropriate amount of water, targeting the desired water to aluminum ratio, was then slowly added by a syringe through the septum, with this addition resulting in immediate heat generation and venting of gases through the vent line. In a typical reaction, addition of the water required about one hour, but this amount of time depended on the size of the batch, the water:aluminum ratio, and the resultant exotherm. Reaction temperatures reached as high as 65° C., but no preferred maximum or minimum exotherm was targeted. Following the water addition, the reaction was allowed to continue stirring until reaching about 30° C., and the reaction mixture was filtered to remove precipitated species. The filtrate was then analyzed by ICP to determine the weight percent aluminum in the final product. By determining the percentage of aluminum in the final product as well as the mass of the aluminoxane solution, it was possible to calculate the percent of “lost” aluminum (insolubles) isolated as precipitate.

The ICP method involved taking a carefully weighed aliquot from the final product, quenching the alkyls with a carefully weighed amount of heavy alcohol, and weighing this combination (some losses occur during quenching). The weight percent aluminum of the quench mixture was then used to back-calculate the percent aluminum in the aluminoxane. It is not recommended to perform ICP experiments on toluene solutions, since the digestion step involves nitric acid. ICP analysis for the amount of aluminum (wt. %) in the composition and aluminum loss (wt. %) utilized a PerkinElmer Optima 8300 instrument.

Examples 22-37 were conducted to determine the amount of aluminum loss, due to the formation of insoluble materials, as a function of the water:aluminum ratio. Table 3 summarizes the experiments of Examples 22-37. The reactions were initiated at room temperature, unless otherwise noted, and the reaction conditions shown in Table 3 include the relative molar amount of TEA:TMA, the molar ratio of water:aluminum, and the hydrocarbon solvent utilized. Table 3 summarizes the final amount of aluminum in the composition and the amount of aluminum loss due to the formation of insoluble materials.

As demonstrated by the data in Table 3, aluminum loss generally increased with an increase in the molar ratio of water:aluminum. While aluminum loss might suggest that it would be better to use a lower water:aluminum ratio, this would need to be balanced with the overall activity of the resulting alkylaluminoxane composition. Also of note, any reduction in the amount of TMA (i.e., more TEA used to produce the alkylaluminoxane) also represents a cost savings. In sum, each of Examples 22-37 produced an excellent alkylaluminoxane composition. In particular, the alkylaluminoxane compositions of Examples 31-37 offer a beneficial combination of properties, with Examples 32-36 providing lower amounts of aluminum loss.

TABLE 3

| | water:Al | Al Lost | Final Al | |
|---|---|---|---|---|
| Example | (mol) | (wt. %) | (wt. %) | Reaction Conditions |
| 22* | 0.8 | 43 | 1.22 | 50:50 TEA:TMA, toluene |
| 23* | 0.8 | 38 | 2.12 | 50:50 TEA:TMA, toluene, 0° C. |
| 24* | 0.4 | 12 | 1.84 | 50:50 TEA:TMA, toluene |
| 25 | 0.6 | 28 | 3.38 | 75:25 TEA:TMA, xylene |
| 26 | 0.6 | 27 | 3.39 | 90:10 TEA:TMA, xylene |
| 27 | 0.6 | 28 | 3.37 | 75:25 TEA:TMA, xylene |
| 28 | 0.68 | 32 | 5.18 | 75:25 TEA:TMA, xylene |
| 29 | 0.68 | 28 | 4.74 | 75:25 TEA:TMA, cyclohexane |
| 30 | 0.68 | 29 | 4.34 | 100 TEA, methyl cyclohexane |
| 31 | 0.6 | 29 | 3.62 | 50:50 TEA:TMA, xylene |
| 32 | 0.5 | 16 | 5.70 | 75:25 TEA:TMA, xylene |
| 33 | 0.4 | 13 | 5.77 | 80:20 TEA:TMA, cyclohexane |
| 34 | 0.4 | 13 | 5.60 | 75:25 TEA:TMA, 1-hexene |
| 35 | 0.4 | 10 | 5.14 | 75:25 TEA:TMA, cyclohexane |
| 36 | 0.5 | 7.7 | 5.56 | 75:25 TEA:TMA, cyclohexane |
| 37 | 0.63 | 21 | 5.00 | 75:25 TEA:TMA, cyclohexane |

* Aluminum loss determined by gravimetric or mass balance method. Other examples utilized ICP analysis to determine aluminum loss.

### Example 38

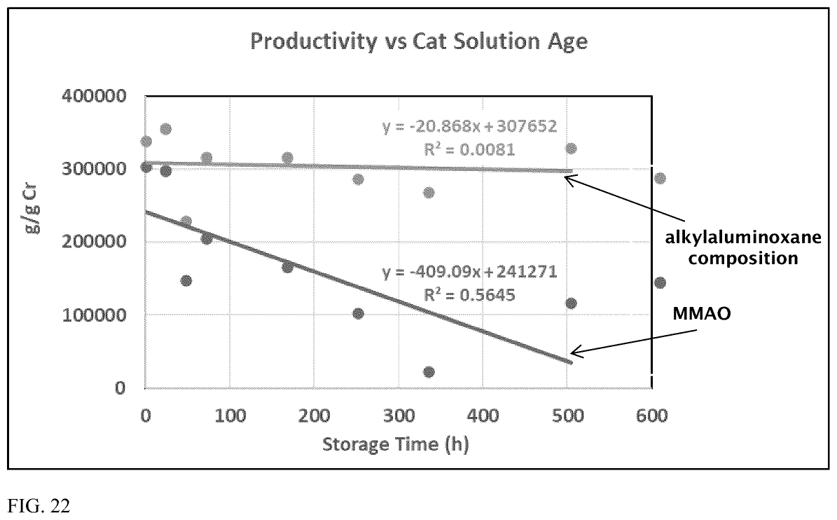

Example 38 demonstrates that catalyst compositions containing the alkylaluminoxane compositions described herein are more stable and have a longer shelf-life (constant oligomerization activity) as compared to analogous catalyst compositions containing MMAO. The results are summarized in FIG. 22.

Chromium catalyst compositions were prepared and ethylene oligomerizations were performed as follows. In a dry box, two glass scintillation vials were charged with equal amounts of a representative heteroatomic ligand chromium compound complex (a N²-phosphinyl guanidine chromium (III) trichloride tetrahydrofuran complex), 10.0 g of ethylbenzene, and 10.0 g of n-nonane (internal standard). After stirring for 30 minutes, MMAO at a 400:1 Al:Cr ratio was added to one of the vials (the control), and the inventive alkylaluminoxane composition of Example 25 (molar ratio of water:Al of 0.6:1, molar ratio of TEA:TMA of 75:25) was added to the second vial, also in a 400:1 Al:Cr ratio. Both vials were allowed to continue stirring for an additional hour, and the contents of each vial were diluted further with cyclohexane to give resultant solution concentrations of 2.0×10⁻⁴ M [Cr]. The solutions were stored in separate 500 mL bottles in the absence of air or moisture.

To test the stored catalyst solutions, 23.4 mL of each solution were pulled at different time intervals and diluted with cyclohexane to 200 mL total volume. The resultant solution was then charged to an evacuated 0.5 L stainless steel reactor heated to 70° C. Hydrogen (50 psig) was charged into the reactor followed by ethylene (875 psig). The reaction proceeded to exotherm to the target temperature of 85° C. with ethylene being fed on-demand to maintain the desired reactor pressure. After 30 min, the oligomerization reaction was rapidly cooled to 30° C., and then the unreacted ethylene and hydrogen gas were vented.

A liquid sample was collected, filtered, and analyzed by gas chromatography, typically using n-nonane as an internal standard, to determine the amount of oligomers produced, and therefore the catalyst activity in grams of oligomer product per gram of chromium. Both the control and the experimental catalyst solutions were tested at 1, 24, 48, and 72 hours, as well as 1, 1.5, 2, and 3 weeks. FIG. 22 shows no loss of productivity in the alkylaluminoxane composition catalyst solution, but significant productivity loss in the control catalyst solution.

The top (flat) line in FIG. 22 is from the series of oligomerization experiments utilizing the alkylaluminoxane composition and the bottom (decreasing) line is from the series of oligomerization experiments utilizing the comparative MMAO activator. Unexpectedly, catalyst compositions containing the alkylaluminoxane compositions disclosed herein had stable catalyst activity for 3 weeks; the catalyst had essentially the same activity for the entire three week test period. This is particularly beneficial in manufacturing operations, where large batches of the mixture can be prepared and stored for extended periods of time. In contrast, the catalyst composition containing the MMAO activator had a significant drop in activity after only 24 hr, and the activity was reduced in half after approximately 1 week.

### Example 39

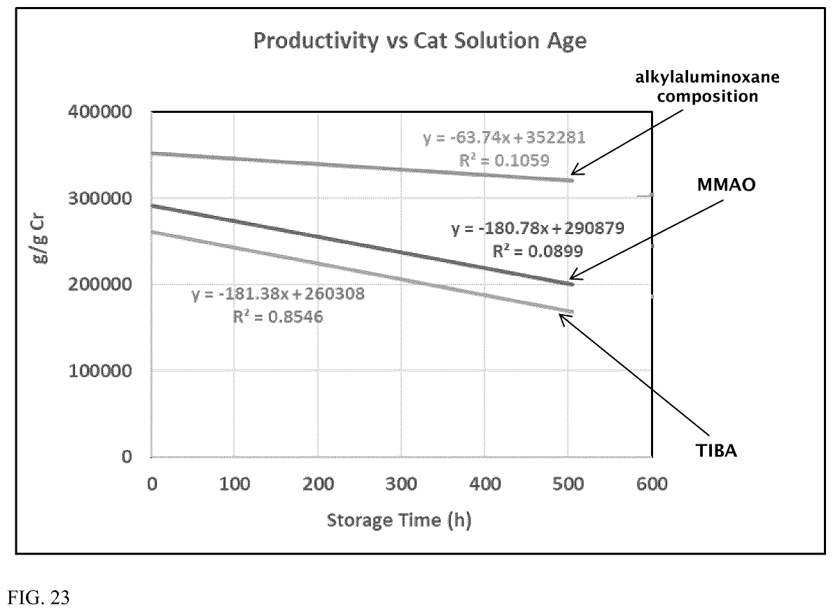

Example 39 demonstrates that catalyst compositions containing the alkylaluminoxane compositions described herein are more stable and have a longer shelf-life (constant oligomerization activity) as compared to analogous catalyst compositions containing MMAO or alkylaluminoxane compositions mixed with TIBA. The results are summarized in FIG. 23.

The experiments of Example 39 were performed similarly to that of Example 28, except the alkylaluminoxane of Example 27 (molar ratio of water:Al of 0.6:1, molar ratio of TEA:TMA of 75:25) was used to prepare the inventive catalyst sample. In addition, a third catalyst solution was prepared with extra TIBA added to the solution containing the chromium complex and the alkylaluminoxane of Example 27. For the experiment with TIBA, TIBA was added to the chromium complex and the alkylaluminoxane composition at a molar ratio of TIBA:Cr of 25:1. Similar to FIG. 22, FIG. 23 also demonstrates the unexpectedly superior stability and catalyst activity of catalyst compositions containing the alkylaluminoxane composition as compared to catalyst compositions containing the comparative MMAO activator. However, the catalyst compositions containing the alkylaluminoxane compositions mixed with TIBA performed worse than that of the inventive catalyst system, and similar to the control MMAO catalyst system. While not wishing to be bound by theory, it is believed that presence of TIBA caused the poor stability and catalyst activity of the catalyst composition containing the alkylaluminoxane composition mixed with TIBA.

### Example 40

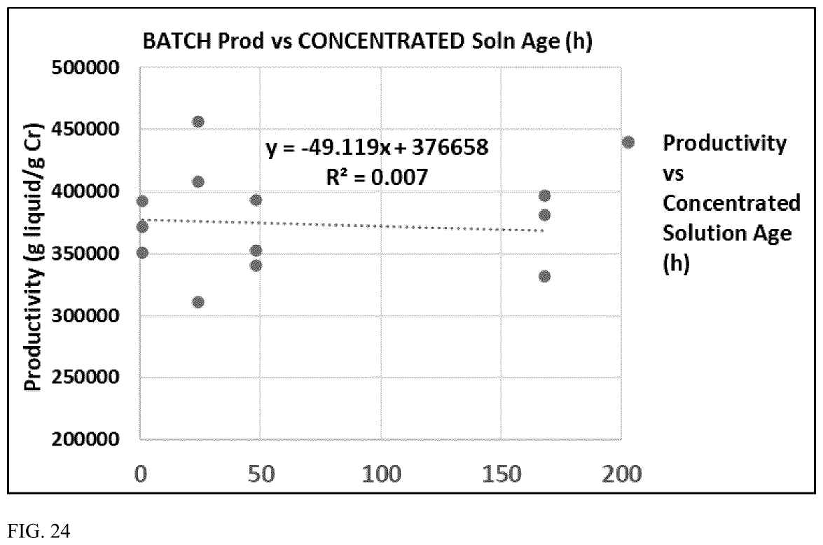

A third lifetime study was carried out, but only the inventive catalyst was prepared. The Cr complex was stirred and activated in the same manner as Example 38, except the alkylaluminoxane composition of Example 28 (molar ratio of water:Al of 0.68:1, molar ratio of TEA:TMA of 75:25) was used to activate the stirred suspension of the Cr complex. This activated catalyst solution was stored at this concentration. No control catalyst solution was prepared, and no additional cyclohexane was added to further dilute the solution to 2.0×10⁻⁴ M [Cr]. The resultant solution had a concentration of about 2.0×10⁻³ M [Cr]. Aliquots of the solution were tested for catalyst productivity at 1, 24, 48, and 168 hours. As demonstrated in FIG. 24, the triplicate run data had no loss in productivity after one week, which shows that even concentrated, activated catalyst solutions possess high stability for long periods of time.

### Examples 41-51

Examples 41-51 were performed in substantially the same manner as that of Examples 1-21, except that an alkylaluminoxane composition was utilized instead of MMAO-3A. Table 4 summarizes the ethylene oligomerization experiments of Examples 41-51. The ligands are shown in Table 1 and the alkylaluminoxane compositions are shown in Table 3.

As shown in Table 4, the alkylaluminoxane compositions of Examples 32-37 were excellent activators for the Fe-based catalyst systems of Examples 41-51. Yields of over 20 g of C₄-C₂₆ products were achieved, as well as catalyst productivities in the range of 10,000-60,000 g/mmol ligand.

Example 44 was run using 1-hexene as the diluent, and 96.8 wt. % purity 1-octene was still obtained, indicating that commercially relevant purities can be obtained under conditions of very high product (i.e., alpha-olefin) concentrations.

TABLE 4

| | | Ligand | Activator | | Tᵢₙᵢₜᵢₐₗ | K | K | Productivity | Yield |
|---|---|---|---|---|---|---|---|---|---|
| | Ligand | MW | Example | Metal source | Tₘₐₓ | (nC₁₂/ | (nC₁₀/ | (g/mmol | (g C4- |
| Example | (mg) | (g/mol) | (Al:Ligand) | (metal:ligand) | (° C.) | nC₁₀) | nC₈) | ligand) | C26) |
| 41 | 2 (0.43) | 432 | 32 (1000:1) | Fe(octanoate)₃ | 30 | 0.770 | 0.747 | 4450 | 4.45 |
| | | | | (4:1) | 50 | | | | |
| 42 | 3 (0.40) | 404 | 32 (1000:1) | Fe(octanoate)₃ | 30 | 0.798 | 0.774 | 2310 | 2.31 |
| | | | | (4:1) | 52 | | | | |
| 43 | 5 (0.46) | 458 | 32 (1000:1) | Fe(octanoate)₃ | 30 | 0.458 | 0.428 | 32,300 | 32.3 |
| | | | | (4:1) | 89 | | | | |
| *44 | 9 (0.43) | 444 | 32 (1000:1) | Fe(octanoate)₃ | 75 | — | 0.437 | 40,460 | 44.8 |
| | | | | (4:1) | 108 | | | | |
| 45 | 7 (0.47) | 470 | 32 (1000:1) | Fe(octanoate)₃ | 30 | 0.472 | 0.447 | 14,600 | 14.6 |
| | | | | (4:1) | 67 | | | | |
| 46 | 8 (0.46) | 456 | 32 (1000:1) | Fe(octanoate)₃ | 30 | 0.200 | 0.151 | 35,700 | 35.7 |
| | | | | (4:1) | 74 | | | | |
| 47 | 9 (0.44) | 444 | 33 (1000:1) | Fe(octanoate)₃ | 30 | 0.508 | 0.500 | 22,300 | 22.3 |
| | | | | (4:1) | 64 | | | | |
| 48 | 10 (0.43) | 430 | 32 (1000:1) | Fe(octanoate)₃ | 30 | 0.345 | 0.213 | 21,400 | 21.4 |
| | | | | (4:1) | 54 | | | | |
| 49 | 11 (0.46) | 458 | 37 (1000:1) | Fe(octanoate)₃ | 30 | — | 0.493 | 61,290 | 60.6 |
| | | | | (4:1) | 100 | | | | |
| 50 | 11 (0.46) | 458 | 36 (1000:1) | Fe(octanoate)₃ | 30 | — | 0.468 | 53,010 | 53.2 |
| | | | | (4:1) | 100 | | | | |
| 51 | 11 (0.46) | 458 | 35 (1000:1) | Fe(octanoate)₃ | 30 | — | 0.512 | 35,710 | 35.9 |
| | | | | (4:1) | 81 | | | | |

### Examples 52-53

Polymerization of 1-hexene using a metallocene catalyst and different activators was evaluated in Examples 52-53. A solution of bis(ethylcyclopentadienyl) zirconium (IV) dichloride (CAS No. 1291-32-3) was prepared from 2.0 mg of the metallocene complex per 1 ml of m-xylene. Two test reaction solutions were prepared using 0.5 mL of the standard metallocene solution (1.0 mg of metallocene) and 20 g of 1-hexene in each reaction vessel.

For Example 52, which served as the control, 0.45 g of a commercially available MMAO was added, the catalyst was activated, and polymerization began to occur, which was confirmed by a reaction exotherm that raised the reaction temperature from room temperature to 57° C. The Al:Zr molar ratio for this reaction was 400:1. The reaction was quenched after 2 hours, and the conversion was determined by GC to be 61%, by using the m-xylene as an internal standard.

For Example 53, an identical procedure was used, except the alkylaluminoxane composition of Example 28 (molar ratio of water:Al of 0.68:1, molar ratio of TEA:TMA of 75:25) was used instead of MMAO. For Example 53, no exotherm was observed following activation. The Al:Zr molar ratio for this reaction was 400:1. The reaction was quenched after 2 hours, and essentially no conversion was observed by GC. This surprising result shows that the alkylaluminoxanes of the invention are not effective for activation of metallocenes, and it was therefore unexpected that they are highly effective at activating other transitional metal based (e.g., Fe-based) catalyst systems.

### Examples 54-58

These examples show continuous steady-state conditions conducted in accordance with the continuous ethylene oligomerization process illustrated in FIG. 1. Each example in Table 5 was run under steady state conditions for at least two residence times, and approximate feed rates are listed for each example. Samples and productivity data also were obtained under steady-state conditions. The ligands are shown in Table 1 and the alkylaluminoxane compositions are shown in Table 3.

For Example 54, 1-dodecene was used during process start-up, then was turned off during steady state operation, demonstrating that the oligomerization process can be operated without added organic reaction medium (mixed C₄⁺ oligomer product functioned as the organic reaction medium). Oligomer distribution and C₈ product purity (99.1%) in Example 54 were unaffected by the use of the oligomer product as the reaction medium.

The K (nC₁₂/nC₁₀) and K (nC₁₀/nC₈) values for Examples 54-58 were around 0.5, as shown by the desirable carbon number distribution in Table 5. Also beneficially, Examples 54-58 exhibited surprisingly high ligand productivity, iron productivity, and aluminum productivity. These productivities are based on g C₄-C₂₆ oligomer product per gram of ligand, iron, and aluminum, respectively, and the respective K values were used to estimate the yield of light olefins. Oligomer carbon number distribution and C8 purity were determined via gas chromatography (FID).

TABLE 5

| | Example | | | | |
|---|---|---|---|---|---|
| | 54 | 55 | 56 | 57 | 58 |
| Ligand | Ligand 9 | Ligand 5 | Ligand 5 | Ligand 7 | Ligand 5 |
| Aluminoxane | 32 | 32 | 32 | 32 | 34 |
| Fe | Fe(Oct)₃ | Fe(Oct)₃ | Fe(Oct)₃ | Fe(Oct)₃ | Fe(Oct)₃ |
| Reaction Medium | 1-dodecene | cyclohexane | cyclohexane | cyclohexane | cyclohexane |
| Reaction Medium | 0 | 210 | 238 | 222 | 280 |
| Feed Rate (g/h) | | | | | |
| Ethylene Feed | 450 | 400 | 350 | 450 | 300 |
| Rate (g/h) | | | | | |
| Residence Time | 45.1 | 33.5 | 34.6 | 30.4 | 35.3 |
| (min, approx.) | | | | | |
| Ligand feed rate | 2 | 1.8 | 1.5 | 1.5 | 1.5 |
| (micromole/h) | | | | | |
| Ligand | 1.91 | 1.31 | 1.13 | 1.04 | 1.15 |
| (ppm in reactor) | | | | | |
| Fe:Ligand (molar) | 3.5 | 3.5 | 3.5 | 3 | 6 |
| Fe (ppm in reactor) | 0.84 | 0.56 | 0.49 | 0.37 | 0.85 |
| Al:Ligand (molar) | 225 | 200 | 300 | 300 | 300 |
| Al (ppm in reactor) | 26.1 | 15.5 | 20 | 17.7 | 20.5 |
| Ethylene | 81.8 | 82.5 | 67.8 | 87.8 | 43.2 |
| Conversion (%) | | | | | |
| Ligand Productivity | 185,000 | 183,000 | 155,000 | 243,000 | 86,000 |
| (g/mmol) | | | | | |
| Fe Prod (g/g) | 939,000 | 935,000 | 792,000 | 1,444,000 | 257,000 |
| Al Prod (g/g) | 30,300 | 34,000 | 19,200 | 30,000 | 10,600 |
| % C4 | 34 | 34 | 34 | 37.8 | 35.2 |
| % C6 | 24 | 24 | 24 | 25.3 | 24.9 |
| % C8 | 15 | 16 | 16 | 15.4 | 15.9 |
| % C10 | 10 | 10 | 10 | 9.0 | 9.8 |
| C8 purity | 99.1 | — | 99.3 | 99.6 | — |
| Temperature (C./F.) | 85 (185) | 85 (185) | 80 (176) | 80 (176) | 80 (176) |

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A catalyst composition comprising (a) an organoaluminum compound, (b) a hydrocarbon diluent, and (c) a heteroatomic ligand transition metal compound complex comprising Fe, Co, or Ni.

Aspect 2. A catalyst composition comprising (A) an organoaluminum compound, (B) a hydrocarbon diluent, (C) a heteroatomic ligand, and (D) a transition metal compound comprising Fe, Co, or Ni, wherein the transition metal compound is soluble in the hydrocarbon diluent.

Aspect 3. An oligomerization process comprising (i) contacting ethylene, an organic reaction medium, and a catalyst composition comprising (I) an organoaluminum compound, a heteroatomic ligand, and a transition metal compound comprising Fe, Co, or Ni, wherein the transition metal compound is soluble in the organic reaction medium, or (II) an organoaluminum compound and a heteroatomic ligand transition metal compound complex comprising Fe, Co, or Ni, in an oligomerization reactor, (ii) forming an oligomer product in the oligomerization reactor, the oligomer product comprising hexenes and octenes, and (iii) discharging an effluent stream from the oligomerization reactor, the effluent stream comprising unreacted ethylene and the oligomer product.

Aspect 4. The process defined in aspect 3, wherein the catalyst composition further comprises a hydrocarbon diluent.

Aspect 5. The process defined in aspect 3 or 4, wherein hydrogen is present in step (i).

Aspect 6. The composition or process defined in any one of aspects 1-5, wherein the organoaluminum compound comprises an aluminoxane, an alkylaluminum compound, or any combination thereof.

Aspect 7. The composition or process defined in aspect 6, wherein the aluminoxane comprises methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or a combination thereof.

Aspect 8. The composition or process defined in aspect 6, wherein the aluminoxane comprises an alkylaluminoxane prepared from trimethylaluminum and triethylaluminum.

Aspect 9. The composition or process defined in aspect 6, wherein the alkylaluminum compound comprises trimethylaluminum, triethylaluminum, tripropylaluminum, tri-n-butylaluminum, tri-isobutylaluminum, trihexylaluminum, tri-n-octylaluminum, diethylaluminum chloride, diethylaluminum bromide, ethylaluminum dichloride, ethylaluminum sesquichloride, or a combination thereof.

Aspect 10. The composition or process defined in any one of aspects 1-9, wherein the hydrocarbon diluent and the organic reaction medium are the same or different and independently comprise a saturated aliphatic hydrocarbon, an aromatic hydrocarbon, a linear α-olefin, or any combination thereof.

Aspect 11. The composition or process defined in aspect 10, wherein the saturated aliphatic hydrocarbon comprises propane, butane, pentane, hexane, heptane, octane, cyclohexane, methyl cyclohexane, or combinations thereof.

Aspect 12. The composition or process defined in aspect 10, wherein the aromatic hydrocarbon comprises benzene, toluene, xylene, cumene, ethylbenzene, or combinations thereof.

Aspect 13. The composition or process defined in aspect 10, wherein the linear α-olefin comprises 1-butene, 1-hexene, 1-octene, 1-decene, 1-dodecene, 1-tetradecene, or combinations thereof.

Aspect 14. The composition or process defined in any one of aspects 1-9, wherein the hydrocarbon diluent and the organic reaction medium are the same or different and independently comprise a linear α-olefin, e.g., 1-butene, 1-hexene, 1-octene, 1-decene, 1-dodecene, 1-tetradecene, or combinations thereof.

Aspect 15. The composition or process defined in any one of aspects 1 and 3-14, wherein the heteroatomic ligand transition metal compound complex (comprising Fe, Co, or Ni) comprises any suitable heteroatomic ligand transition metal compound complex or any heteroatomic ligand transition metal compound complex disclosed herein.

Aspect 16. The composition or process defined in any one of aspects 1 and 3-14, wherein the heteroatomic ligand transition metal compound complex comprising Fe, Co, or Ni comprises a phosphine-imino-quinoline ligand.

Aspect 17. The composition or process defined in any one of aspects 2-14, wherein the heteroatomic ligand comprises any suitable heteroatomic ligand or any heteroatomic ligand disclosed herein.

Aspect 18. The composition or process defined in any one of aspects 2-14, wherein the heteroatomic ligand comprises a phosphine-imino-quinoline ligand.

Aspect 19. The composition or process defined in any one of aspects 2-14 and 17-18, wherein the transition metal compound has the formula M(X¹)ₚ, wherein M is Fe, Co, or Ni, p is an oxidation state of M, and each X¹ independently is a monoanionic ligand.

Aspect 20. The composition or process defined in aspect 19, wherein each X¹ independently is a halogen, a carboxylate, a β-diketonate, a hydrocarboxide, a nitrate, or a chlorate.

Aspect 21. The composition or process defined in aspect 19, wherein each X¹ independently is acetate, a propionate, a butyrate, a pentanoate, a hexanoate, a heptanoate, an octanoate, a nonanoate, a decanoate, an undecanoate, a dodecanoate, or acetylacetonate.

Aspect 22. The composition or process defined in any one of aspects 1-21, wherein a molar ratio of Al:transition metal (or Al:ligand) in the catalyst composition is in any range disclosed herein, e.g., from 10:1 to 5,000:1, from 50:1 to 3,000:1, from 75:1 to 2,000:1, from 100:1 to 2,000:1, or from 100:1 to 1,000:1.

Aspect 23. The composition or process defined in any one of aspects 2-22, wherein a molar ratio of ligand:transition metal in the catalyst composition is in any range disclosed herein, e.g., from 20:1 to 1:20, from 10:1 to 1:10, from 8:1 to 1:8, from 5:1 to 1:5, from 4:1 to 1:4, or from 2:1 to 1:2.

Aspect 24. The process defined in any one of aspects 3-23, wherein the oligomerization reactor comprises any suitable reactor type, e.g., a continuous stirred tank reactor, a loop reactor, or any combination thereof.

Aspect 25. The process defined in any one of aspects 3-24, wherein ethylene is introduced into the reactor separately from the catalyst composition (or catalyst system components).

Aspect 26. The process defined in any one of aspects 3-25, wherein the catalyst composition is formed and then introduced into the oligomerization reactor.

Aspect 27. The process defined in any one of aspects 3-25, wherein the catalyst composition is formed within the oligomerization reactor.

Aspect 28. The process defined in any one of aspects 3-25, wherein the organoaluminum compound, the heteroatomic ligand, and the transition metal compound are introduced separately into the oligomerization reactor.

Aspect 29. The process defined in any one of aspects 3-25, wherein at least a portion of the heteroatomic ligand and at least a portion the transition metal compound are combined to form at least a portion of the heteroatomic ligand transition metal compound complex before being introduced into the oligomerization reactor.

Aspect 30. The process defined in any one of aspects 3-25, wherein the organic reaction medium and the organoaluminum compound are combined and then introduced into the oligomerization reactor.

Aspect 31. The process defined in any one of aspects 3-30, wherein the oligomer product comprises any amount of octenes disclosed herein, e.g., at least 5, 10, 20, 30 or 40 wt. %; a maximum of 99, 95, 92.5, 90, 87.5, or 85 wt. %; or from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes, based on the total amount of oligomers in the oligomer product.

Aspect 32. The process defined in any one of aspects 3-31, wherein the oligomer product comprises any amount of hexenes disclosed herein, e.g., at least 10, 15, 20, 25, 30, or 35 wt. %; a maximum of 75, 65, 60, 55, or 50 wt. %; or from 10 to 75 wt. %, from 15 to 65 wt. %, from 20 to 60 wt. %, from 25 to 55 wt. %, or from 30 to 50 wt. % hexenes, based on the total amount of oligomers in the oligomer product.

Aspect 33. The process defined in any one of aspects 3-32, wherein the oligomerization reactor has any ethylene conversion disclosed herein, e.g., at least 20, 30, 35, 40, 45, or 50 wt. %; a maximum of 99, 95, 90, 80, 75, 70, or 65 wt. %; or from 20 to 95 wt. %, from 30 to 90 wt. %, from 40 to 80 wt. %, from 50 to 70 wt. %, or from 55 to 65 wt. % conversion, based on the amount of ethylene entering the reactor and the amount of ethylene in the effluent stream.

Aspect 34. The process defined in any one of aspects 3-33, wherein the oligomer product is formed at any suitable oligomerization temperature and oligomerization pressure or any oligomerization temperature and pressure disclosed herein.

Aspect 35. The process defined in any one of aspects 3-34, further comprising a step of contacting the effluent stream with a catalyst system deactivating agent.

Aspect 36. The process defined in any one of aspects 3-35, further comprising a step of separating/isolating a C₆ stream comprising any amount of hexene(s) disclosed herein, e.g., at least 96, 97, 98, or 99 wt. % hexene(s) from the oligomer product, and/or a step of separating/isolating a C₈ stream comprising any amount of octene(s) disclosed herein, e.g., at least 96, 97, 98, or 99 wt. % octene(s) from the oligomer product.

Aspect 37. The process defined in aspect 36, wherein the C₈ stream comprises any amount of 1-octene disclosed herein, e.g., at least 95, 96, 96.5, 97, 97.5, 98, or 98.5 wt. % 1-octene, based on the total weight of the octene(s).

Aspect 38. The process defined in aspect 36 or 37, wherein the C₆ stream comprises any amount of 1-hexene disclosed herein, e.g., at least 90, 92.5, 95, 97.5, 98, 98.5, or 99 wt. % 1-hexene, based on the total weight of the hexene(s).

What is claimed is:

1. An oligomerization process comprising:
(i) contacting ethylene, an organic reaction medium, and a catalyst composition comprising an organoaluminum compound, a heteroatomic ligand, and a transition metal compound comprising Fe, Co, or Ni, in an oligomerization reactor;
(ii) forming an oligomer product in the oligomerization reactor, the oligomer product comprising hexenes and octenes; and
(iii) discharging an effluent stream from the oligomerization reactor, the effluent stream comprising unreacted ethylene and the oligomer product.

2. The process of claim 1, wherein the catalyst composition further comprises a hydrocarbon diluent.

3. The process of claim 2, wherein the hydrocarbon diluent and the organic reaction medium are the same or different and independently comprise a saturated aliphatic hydrocarbon, an aromatic hydrocarbon, a linear α-olefin, or any combination thereof.

4. The process of claim 2, wherein the transition metal compound is soluble in the hydrocarbon diluent, or the transition metal compound is soluble in the organic reaction medium, or both.

5. The process of claim 1, wherein the heteroatomic ligand is a compound having the following formula: 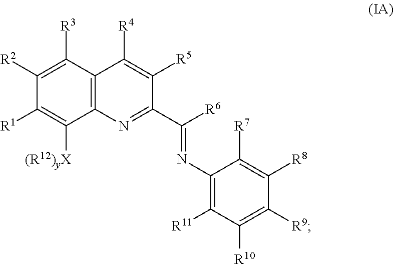
wherein:
X is P or S;
y is equal to 1 when X is S, and y is equal to 2 when X is P;
R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and
each R¹² independently is a C₁-C₁₈ hydrocarbyl group or a C₁-C₁₈ halogenated hydrocarbyl group.

6. The process of claim 1, wherein the heteroatomic ligand is a compound having the following formula: 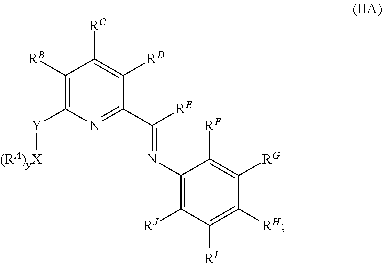
wherein:
X is P or S;
y is equal to 1 when X is S, and y is equal to 2 when X is P;
Y is O, NH, or CH₂;
R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and
each R^{A} independently is a C₁-C₁₈ hydrocarbyl group or a C₁-C₁₈ halogenated hydrocarbyl group.

7. The process of claim 1, wherein the transition metal compound has the formula M(X¹)ₚ, wherein:
M is Fe, Co, or Ni;
p is an oxidation state of M; and
each X¹ independently is a monoanionic ligand.

8. The process of claim 7, wherein:
M is Fe; and
each X¹ independently is acetate, a propionate, a butyrate, a pentanoate, a hexanoate, a heptanoate, an octanoate, a nonanoate, a decanoate, an undecanoate, a dodecanoate, or acetylacetonate.

9. The process of claim 1, wherein:
a molar ratio of ligand:transition metal in the catalyst composition is in a range from 20:1 to 1:20; and/or
a molar ratio of Al:ligand in the catalyst composition is in a range from 10:1 to 5,000:1.

10. The process of claim 1, wherein the organoaluminum compound comprises an aluminoxane comprising methylaluminoxane (MAO), ethylaluminoxane, modified methylaluminoxane (MMAO), n propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or a combination thereof.

11. The process of claim 1, wherein the organoaluminum compound comprises an alkylaluminoxane composition comprising:
(I) an alkylaluminoxane having random repeating units of formula (A) and formula (B) 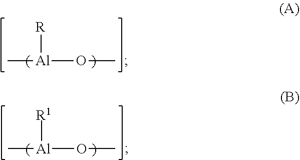
wherein:
R is methyl and R¹ is ethyl at a molar ratio of methyl:ethyl from 5:95 to 80:20; and
(II) a hydrocarbon solvent;
wherein an amount of aluminum in the composition is from 0.1 to 20 wt. %.

12. The process of claim 1, wherein:
the organoaluminum compound comprises an alkylaluminoxane composition; and
prior to step (i), reacting trimethylaluminum (TMA), triethylaluminum (TEA), and water in a hydrocarbon solvent to form an alkylaluminoxane, wherein a molar ratio of TMA:TEA is from 5:95 to 80:20, and a molar ratio of water:Al is from 0.2:1 to 1:1; and removing insoluble aluminum-containing materials from the solvent to form the alkylaluminoxane composition containing from 0.1 to 20 wt. % of aluminum.

13. The process of claim 1, wherein the catalyst composition is formed and then introduced into the oligomerization reactor.

14. The process of claim 1, wherein the catalyst composition is formed within the oligomerization reactor.

15. The process of claim 1, wherein the oligomerization reactor comprises a continuous stirred tank reactor, a loop reactor, or any combination thereof.

16. The process of claim 1, wherein at least a portion of the heteroatomic ligand and at least a portion of the transition metal compound are combined to form at least a portion of a heteroatomic ligand transition metal compound complex before being introduced into the oligomerization reactor.

17. The process of claim 1, wherein:
hydrogen is present in step (i) of the oligomerization process;
ethylene is introduced into the oligomerization reactor separately from the catalyst composition or catalyst system components;
the organic reaction medium and the organoaluminum compound are combined and then introduced into the oligomerization reactor; or
any combination thereof.

18. An oligomerization process comprising:
(i) contacting ethylene, an organic reaction medium, and a catalyst composition comprising an organoaluminum compound and a heteroatomic ligand transition metal compound complex comprising Fe, Co, or Ni, in an oligomerization reactor;
(ii) forming an oligomer product in the oligomerization reactor, the oligomer product comprising hexenes and octenes; and
(iii) discharging an effluent stream from the oligomerization reactor, the effluent stream comprising unreacted ethylene and the oligomer product.

19. A catalyst composition comprising:
(A) an organoaluminum compound;
(B) a hydrocarbon diluent;
(C) a heteroatomic ligand; and
(D) a transition metal compound comprising Fe, Co, or Ni.

20. A catalyst composition comprising:
(a) an organoaluminum compound;
(b) a hydrocarbon diluent; and
(c) a heteroatomic ligand transition metal compound complex comprising Fe, Co, or Ni.
